(19) United States
(12) Patent Application Publication
(10) Pub. No.: US 2020/0225244 A1
(43) Pub. Date: Jul. 16, 2020

(54) COMPOSITIONS AND METHODS FOR ASSESSING PAINFUL DEMYELINATING AND NONDEMYELINATING DISEASES

(71) Applicants: Veronica Shubayev, La Jolla, CA (US); Sanford Burnham Prebys Medical Discovery Institute, La Jolla, CA (US)

(72) Inventors: Veronica Shubayev, La Jolla, CA (US); Alex Strongin, La Jolla, CA (US); Albert G. Remacle, La Jolla, CA (US)

(21) Appl. No.: 16/651,114

(22) PCT Filed: Sep. 25, 2018
(86) PCT No.: PCT/US2018/052565

§ 371 Date: Mar. 26, 2020

Related U.S. Application Data
(60) Provisional application No. 62/563,347, filed on Sep. 26, 2017.

(51) Int. Cl. G01N 33/68; G01N 33/543; A61K 31/197; A61P 25/00

(52) U.S. Cl. CPC G01N 33/6854; G01N 33/543; A61K 31/197; A61K 45/06; G01N 2800/285; G01N 2800/52; A61P 25/00

(57) ABSTRACT

Disclosed are compositions, kits, and methods for detecting and assessing demyelinating diseases and conditions, neuropathic pain related to demyelinating diseases and conditions, selecting subjects for treatment with a ligand for voltage-gated Ca²⁺-channel α2δ1 (CACNA2D1 ligand), and selecting subjects to not be treated with a CACNA2D1 ligand. It has been discovered that the presence of antibodies to a proteolytic fragment of myelin basic protein (myelin basic protein-derived peptide (MBP84-104)) in subjects suffering from neuropathic pain indicates that (1) the subject is suffering from a demyelinating disease or condition and (2) that such subjects are more effectively treated with a CACNA2D1 ligand such as gabapentin or pregabalin as distinct from treatment with other pain relievers such as COX inhibitors (such as ketorolac), sodium channel blockers (such as lidocaine), and NMDA antagonists (such as MK801).

### CROSS-REFERENCE TO RELATED APPLICATION

This application claims the benefit of and priority to U.S. Provisional Application No. 62/563,347 filed Sep. 26, 2017, which is hereby incorporated herein by reference in its entirety.

### STATEMENT REGARDING FEDERALLY SPONSORED RESEARCH

This invention was made with Government support under Grant No. 1R01DE022757 awarded by the National Institutes of Health. The Government has certain rights in the invention.

### FIELD OF THE INVENTION

The disclosed invention is generally in the fields of molecular medicine and neurobiology and, specifically in the areas of painful, neuropathic, and demyelinating diseases.

### BACKGROUND OF THE INVENTION

Diseases of the nervous system involving damage to the myelin sheath of neurons are generalized herein as demyelinating diseases. The damage to the myelin sheath impairs the conduction of signals in the affected nerves, and, depending on which nerves are involved, in turn causes deficiencies in sensation, movement, cognition, or other functions.

Demyelinating diseases may be caused by genetics, infectious agents, autoimmune reactions, trauma, toxic chemicals, and other, unknown factors. For example, organophosphates, a class of chemicals which are the active ingredients in commercial insecticides such as sheep dip, weed-killers, and flea treatment preparations for pets, etc., will also demyelinate nerves. Neuroleptics can also cause demyelination (Konopaske et al., Biol. Psychiatry. 63(8):759-65, (2008)).

There are traditionally two classes of demyelinating diseases: demyelinating myelinoclastic diseases and demyelinating leukodystrophic diseases. In the first group a normal and healthy myelin is destroyed by a toxic, chemical or autoimmune substance. The second group, in which myelin is abnormal and degenerates (Fernandez et al., Medicine. 11(77):4601-4609, (2015)), was eventually renamed dysmyelinating diseases (Poser, Arch Neurol. 4(3):323-332, (1961)). The release of myelin autoantigens may occur in the absence of demyelinating diseases.

There is good evidence that multiple sclerosis (MS), the best known example of a demyelinating disease, is of autoimmune origin. Acquired immune system cells called T-cells are known to be present at the site of sclerotic lesions. Other immune system cells called macrophages (and possibly mast cells as well) also contribute to the damage. Additionally, vitamin B12 deficiency has been shown to cause demyelination (Miller et al. J Neurol Sci 233(1-2):93-97, (2005)).

Myelin basic protein (MBP), a major component of the myelin sheath, is involved in the process of myelination of nerves in the nervous system. MBP is encoded by the Golli (genes of oligodendrocyte lineage)-MBP gene in myelinating glia and immune cells (Boggs, Cell Mol Life Sci 63(17):1945-1961, (2006)) and encodes four independent MBP isoforms (18.5 kDa, 17 kDa, 20.2 kDa and 21.5 kDa) (Campagnoni et al. J Biol Chem. 268:4930-4938, (1993); Feng, Neurochem Res. 32:273-278, (2007)). Following mRNA transport to the myelin compartment, neuronal MBP is translated locally (Muller et al. Front Cell Neurosci. 7:169, (2013)). MBP continually changes conformation as a result of its local disorder-to-order transitions (Harauz et al. Micron. 35:503-542, (2004); Harauz et al. Biochemistry 48:8094-8104, (2009); Harauz and Libich, Curr Protein Pept Sci. 10:196-215, (2009); Zhang et al. J Proteome Res. 11:4791-4802, (2012)). As an intrinsically unstructured and positively charged protein with the isoelectric point at pH 10, MBP interacts with the acidic head groups of the lipid bilayer and a variety of polyanionic proteins, including actin, tubulin and Ca²⁺-calmodulin. These interactions regulate multiple functions of the axon-glia unit, including cytoskeletal assembly, Ca²⁺ homeostasis and a protein:lipid ratio in the myelin membranes (Boggs, Cell Mol Life Sci 63(17):1945-1961, (2006); Harauz and Boggs, J Neurochem 215(3):334-361, (2013)).

Interest in MBP has centered on its role in demyelinating diseases such as MS. In experimental models of focal painful nerve injury, including chronic constriction injury (CCI) of rat sciatic nerve, matrix metalloproteinases (MMPs), especially pro-inflammatory MMP-9, degrade MBP and release its algesic, cryptic immunodominant epitopes hidden in the native MBP fold MBP (Kim et al. PLoS One 7:e33664, (2012); Liu et al. J Neuroinflammation 9:119, (2012); Chandler et al. Neurosci Lett 201:223-226, (1995); Chandler et al. Biochem Biophys Res Commun 228:421-429, (1996); D'Souza and Moscarello, Neurochem Res 31:1045-1054, (2006); Gijbels et al. J Neurosci Res 36:432-440, (1993); Proost et al. Biochem Biophys Res Commun. 192:1175-1181, (1993); Shiryaev et al. J Biol Chem. 284:30615-30626, (2009); Shiryaev et al. PLoS One 4:e4952, (2009); Shubayev and Myers, Brain Res. 855:83-89, (2000)). The resulting algesic, immunodominant MBP fragments directly contribute to severe pain hypersensitivity to light tactile stimulation, a phenomenon known as mechanical allodynia. The evolutionary conserved centrally located cryptic MBP epitopes such as the 84-104 region of MBP (MBP84-104, residues are numbered according to the GenBank # AAH08749) (FIG. 1A), when released by proteolysis, are encephalitogenic in patients with MS and in experimental autoimmune encephalomyelitis animals (Boggs, Cell Mol Life Sci 63(17):1945-1961, (2006)). Studies have shown that a localized injection of the MBP84-104 peptide into the intact peripheral nervous system (sciatic nerve) is sufficient to initiate a molecular cascade leading to robust mechanical allodynia in rats (Liu et al., J Neuroinflamm 9:119, (2012)). Because T cell activity is required mainly for the maintenance of MBP84-104-induced allodynia—as athymic nude rats initially develop mild mechanical hypersensitivity after MBP84-104 injection (Liu et al., J Neuroinflamm 9:119, (2012))—and because T cells are among the last immune cell type to infiltrate the peripheral nervous system injury (Kim and Moalem-Taylor, Brain Res 1405:95-108, (2011b)), the early algesic mechanisms of the MBP84-104 action, preceding or independent of T cell recruitment, have been obscure.

In humans the MMP family consists of eighteen soluble and six membrane-tethered proteases synthesized as zymogens (Egeblad and Werb, Nat Rev Cancer. 2:161-174, (2002)). Soluble MMPs proenzyme contain an N-terminal inhibitory prodomain followed by an active site catalytic domain, a flexible linker region and a C-terminal hemopexin domain. Zymogens require proteolytic removal of their inhibitory prodomain to generate the catalytically active proteases (Egeblad and Werb, Nat Rev Cancer. 2:161-174, (2002)). Once activated, MMP activity is regulated by their four natural inhibitors, tissue inhibitors of metalloproteases (TIMPs), each comprised of the N-terminal inhibitory and the C-terminal non-inhibitory domains. TIMP-1 is the most efficient inhibitor of the pro-inflammatory MMP-9 gelatinase (Brew and Nagase, Biochim Biophys Acta. 1803:55-71, (2010)). TIMP-1 via its C-terminal domain also forms a unique stoichiometric complex (1:1), stable heterodimer with the hemopexin domain of MMP-9 proenzyme. This complex is significantly more resistant to activation relative to the TIMP-1-free MMP-9 proenzyme (Goldberg et al. J Biol Chem. 267:4583-4591, (1992)). Both MMP-9 and TIMP-1 are highly up-regulated in the damaged peripheral nervous system (Kim et al. PLoS One 7:e33664, (2012); Chernov et al. J Biol Chem. 290:11771-11784, (2015)) where the enhanced MMP activity plays a cardinal role in immune cell infiltration, Schwann cell activity, demyelination and pain signaling (Hong et al. Brain Behav Immun. 60:282-292, (2017); Chattopadhyay and Shubayev, Glia. 57:1316-1325, (2009); Kobayashi et al. Mol Cell Neurosci. 39:619-627, (2008); Liu et al. J Neuropathol Exp Neurol. 69:386-395, (2010); Shubayev et al. Mol Cell Neurosci. 31:407-415, (2006)). The upregulation of MMP activity in the injured nerve microenvironment and the subsequent proteolytic release of the cryptic immunodominant MBP epitope are likely to be followed by stimulation of autoimmune response (Vargas et al. Proc Natl Acad Sci USA. 107:11993-11998, (2010)).

Besides inducing unilateral allodynia, intrasciatic administration of MBP84-104 has also been shown to increase unilateral IL-6 along the injected neuraxis and especially in the spinal cord. The IL-6 expression patterns after intrasciatic administration of MBP84-104 are highly consistent with those observed in peripheral nervous system injury models, apparent in endoneurial Schwann cells and macrophages (Kurek et al., Neuromuscul Discord 6(2):105-114, (1996); Bolin et al., J Neurochem 64(2):850-858, (1995)), dorsal root ganglia neurons and satellite cells (Dubovy et al., Neuron Glia Biol 6(1):73-83, (2010)), spinal neurons (DeLeo et al., J Interferon Cytokine Res 16(9):695-700, (1996); Arruda et al., Brain Res Mol Brain Res 62(2):228-235, (1998)) and spinal astrocytes (Whitehead et al., Brain Behav Immun 24(4):569-576, (2010)). Similarly to peripheral nervous system injury, intrasciatic MBP84-104 activates the adaptive immune pathways and MHCII expression in the spinal cord (Liu et al., J Neuroinflamm 9:119, (2012); Sweitzer et al., J Neuroimmunol 125(1-2):82-93, (2002)). Although broad degenerative changes in MBP84-104 injected nerves are absent (Liu et al., J Neuroinflamm 9:119, (2012)) and the major pro-inflammatory cytokine expression is unchanged relative to the scrambled peptide, their role in MBP-induced allodynia cannot be ruled out. Increase in IL-6 expression and the satellite cell activation suggest a trophic response in dorsal root ganglia at least partly comparable to peripheral nervous system injury.

MBP displays direct neuron-specific (but not glial) toxicity in vitro, which seems to depend on its binding to sialic acid containing lipids on the neuronal surface and regulation of the nonselective cation flow (Zhang et al., PLoS ONE 9(9):e108646, (2014); Gahwiler and Honegger, Neurosci Lett 11(3):317-321, (1979)). Both in the presence and absence of T cells, intrasciatic MBP84-104 induces IL-6 and spinal Ca²⁺ signaling (Liu et al., J Neuroinflamm 9:119, (2012)). Accordingly, gabapentin reverses MBP84-104-induced pain, by binding voltage-gated Ca²⁺ channel a2d1 (Takasusuki and Yaksh, Anesthesiology 115(1):153-164, (2011)). MBP has also been shown to regulate activity of voltage-gated Ca²⁺ channel and Ca²⁺ flux in oligodendrocytes (Paez et al., J Neurosvi 27(46):12690-12699, (2007); Smith et al., J Neurosci Res 89(4):467-480, (2011)) via a binding to Ca²⁺-calmodulin (Boggs, Cell Mol Life Sci 63(17):1945-1961, (2006)). Through the activity of Ca²⁺-calmodulin-dependent protein kinase, a2d1 controls IL-6 expression in neurons (Sallmann et al., J Neurosci 20(23):8637-8642, (2000)). Although it was conceivable that IL-6 could mediate MBP-induced nociceptive processing by regulating the neuronal a2d1 expression, and Ca²⁺-related excitotoxicity (Spooren et al., Brain Res Rev 67(1-2):157-183, (2011)), the mechanisms of MBP-induced IL-6 expression have not been known. Specifically, how the peripheral changes in large afferent function post-intrasciatic MBP84-104 administration lead to a sustained pain state in response to low threshold mechanical stimuli had been obscure, particularly given the intrinsic inhibition of A-afferents in the dorsal horn. It had also not been understood whether autoantibodies against the algesic region(s) of MBP significantly contribute to painful neuropathy.

There are currently no reliable methods for the diagnosis of chronic neuropathic pain. Consequently, there are no reliable methods of specific, diagnosis-based treatment.

It is an object of the invention to provide kits for detecting antibodies to MBP-derived peptides.

It is a further object of the invention to provide improved methods of objectively detecting the existence of painful neuropathy and demyelination in a subject.

It is a further object of the invention to provide methods of determining the proper course of treatment of painful neuropathy and demyelinating disease.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF SUMMARY OF THE INVENTION

Disclosed are compositions, kits, and methods for detecting and assessing chronic pain states, neuropathic pain related to demyelinating diseases and conditions, demyelinating diseases and conditions, selecting subjects for treatment with a ligand for voltage-gated Ca²⁺-channel α2δ1 (CACNA2D1 ligand), and selecting subjects to not be treated with a CACNA2D1 ligand. It has been discovered that the presence of antibodies to a proteolytic fragment of myelin basic protein (myelin basic protein-derived peptide (MBP84-104)) in subjects suffering from neuropathic pain indicates that (1) the subject is suffering from a demyelinating disease or condition and (2) that such subjects are more effectively treated with a CACNA2D1 ligand such as gabapentin or pregabalin as distinct from treatment with other pain relievers such as COX inhibitors (such as ketorolac), sodium channel blockers (such as lidocaine), NMDA antagonists (such as MK801), nonsteroidal anti-inflammatory drugs (NSAIDs) and opiates. These discoveries facilitate avoidance of unnecessary prescription of opioids, NSAIDS, and other problematic pain relievers in addition to the selection of an appropriate treatment.

Disclosed are methods involving treating a subject with a composition consisting essentially of a ligand for voltage-gated Ca²⁺-channel α2δ1 (CACNA2D1 ligand), where antibodies to myelin basic protein-derived peptide (MBP84-104) have been detected in the subject. Also disclosed are methods involving treating a subject with a composition CACNA2D1 ligand, where antibodies to MBP84-104 have been detected in the subject. Also disclosed are methods involving treating a subject with a CACNA2D1 ligand, where antibodies to MBP84-104 were detected in the subject.

In some forms, the CACNA2D1 ligand can be in an effective amount in the composition. In some forms, the composition can include the CACNA2D1 ligand. In some forms, the composition can comprise the CACNA2D1 ligand. In some forms, the composition can consist essentially of the CACNA2D1 ligand. In some forms, the composition can include an effective amount of the CACNA2D1 ligand. In some forms, the composition can comprise an effective amount of the CACNA2D1 ligand. In some forms, the composition can consist essentially of an effective amount of the CACNA2D1 ligand.

In some forms, the methods can further comprise detecting the antibodies to MBP84-104 in the subject prior to treating the subject with the composition. In some forms, the methods can further comprise, prior to treating, detecting antibodies to MBP84-104 in the subject. In some forms, the composition does not include a COX inhibitor, a sodium channel blocker, an NMDA antagonist, an opioid, or a non-steroidal anti-inflammatory drug (NSAID). In some forms, the composition further comprises one or more pain relievers. In some forms, the subject is not treated with a COX inhibitor, a sodium channel blocker, an NMDA antagonist, an opioid, or an NSAID. In some forms, the subject is further treated with one or more pain relievers.

Also disclosed are methods involving detecting antibodies to MBP84-104 in a subject, and treating the subject in which antibodies to MBP84-104 are detected with a CACNA2D1 ligand.

Also disclosed are methods involving treating a subject with a composition consisting essentially of an effective amount of a CACNA2D1 ligand, where antibodies to MBP84-104 were detected in the subject, and where the composition does not include a COX inhibitor, a sodium channel blocker, an NMDA antagonist, an opioid, or an NSAID. Also disclosed are methods involving detecting antibodies to MBP84-104 in a subject, and treating the subject in which antibodies to MBP84-104 are detected with a composition consisting essentially of an effective amount of a CACNA2D1 ligand, where the composition does not include a COX inhibitor, a sodium channel blocker, an NMDA antagonist, an opioid, or an NSAID. Also disclosed are methods involving treating a subject with a CACNA2D1 ligand, where antibodies to MBP84-104 were detected in the subject, and where the composition does not include a COX inhibitor, a sodium channel blocker, an NMDA antagonist, an opioid, or an NSAID. Also disclosed are methods involving detecting antibodies to MBP84-104 in a subject, and treating the subject in which antibodies to MBP84-104 are detected with a CACNA2D1 ligand, where the composition does not include a COX inhibitor, a sodium channel blocker, an NMDA antagonist, an opioid, or an NSAID.

Also disclosed are methods involving treating a subject with a composition consisting essentially of an effective amount of a CACNA2D1 ligand, where antibodies to MBP84-104 were detected in the subject, and where the subject is not treated with a COX inhibitor, a sodium channel blocker, an NMDA antagonist, an opioid, or an NSAID. Also disclosed are methods involving detecting antibodies to MBP84-104 in a subject, and treating the subject in which antibodies to MBP84-104 are detected with a composition consisting essentially of an effective amount of a CACNA2D1 ligand, where subject is not treated with a COX inhibitor, a sodium channel blocker, an NMDA antagonist, an opioid, or an NSAID. Also disclosed are methods involving treating a subject with a CACNA2D1 ligand, where antibodies to MBP84-104 were detected in the subject, and where the subject is not treated with a COX inhibitor, a sodium channel blocker, an NMDA antagonist, an opioid, or an NSAID. Also disclosed are methods involving detecting antibodies to MBP84-104 in a subject, and treating the subject in which antibodies to MBP84-104 are detected with a CACNA2D1 ligand, where subject is not treated with a COX inhibitor, a sodium channel blocker, an NMDA antagonist, an opioid, or an NSAID.

Also disclosed are methods involving treating a subject with a composition consisting essentially of an effective amount of a CACNA2D1 ligand and one or more pain relievers, where antibodies to MBP84-104 were detected in the subject. Also disclosed are methods involving detecting antibodies to MBP84-104 in a subject, and treating the subject in which antibodies to MBP84-104 are detected with a composition consisting essentially of an effective amount of a CACNA2D1 ligand and one or more pain relievers. Also disclosed are methods involving treating a subject with a CACNA2D1 ligand and one or more pain relievers, where antibodies to MBP84-104 were detected in the subject. Also disclosed are methods involving detecting antibodies to MBP84-104 in a subject, and treating the subject in which antibodies to MBP84-104 are detected with a CACNA2D1 ligand and one or more pain relievers. In some forms, the pain relievers can be COX inhibitors, sodium channel blockers, NMDA antagonists, opioids, NSAIDs, or combinations thereof.

In some forms, detection of an antibody to MBP84-104 in the subject indicates that the subject as has a disease or condition that causes, or is associated with, the presence of, demyelination or neuropathic pain. In some forms, the disease or condition is a demyelinating myelinoclastic disease or a demyelinating leukodystrophic disease. In some forms, the disease or condition is inflammatory demyelination, viral demyelination, acquired metabolic demyelination, hypoxic-ischemic demyelination, or compression-induced demyelination. In some forms, the disease or condition is diabetic neuropathy, shingles, post herpetic neuralgia, neuromas, phantom limb pain, trigeminal neuralgia, multiple sclerosis, acute multiple sclerosis, neuromyelitis optica, concentric sclerosis, acute-disseminated encephalonyelitis, acute hemorrhagic leucoencephalitis, progressive multifocal leucoencephalopathy, human immunodeficiency virus infection, subacute sclerosing panencephalitis, central pontine myelinlysis, extrapontine myelinolysis, fibromyalgia, or complex regional pain syndrome.

In some forms, the subject is suffering allodynia. In some forms, the subject is female. In some forms, the CACNA2D1 ligand is gabapentin or pregabalin.

Also disclosed are methods involving treating a subject with a pain reliever other than a CACNA2D1 ligand, where antibodies to MBP84-104 were not detected in the subject. In some forms, the method further comprises, prior to treating, detecting the absence of antibodies to MBP84-104 in the subject. Also disclosed are methods involving detecting the absence of antibodies to MBP84-104 in a subject, and treating the subject with a pain reliever other than a CACNA2D1 ligand, where antibodies to MBP84-104 are not detected in the subject.

Also disclosed are methods involving refraining from treating a subject with a composition consisting essentially of an effective amount of a CACNA2D1 ligand, where antibodies to MBP84-104 were not detected in the subject. In some forms, the method further comprises, prior to refraining from treating, detecting the absence of antibodies to MBP84-104 in the subject. Also disclosed are methods involving detecting the absence of antibodies to MBP84-104 in a subject, and refraining from treating the subject with a composition consisting essentially of an effective amount of a CACNA2D1 ligand, where antibodies to MBP84-104 are not detected in the subject. Also disclosed are methods involving refraining from treating a subject with a CACNA2D1 ligand, where antibodies to MBP84-104 were not detected in the subject. Also disclosed are methods involving detecting the absence of antibodies to MBP84-104 in a subject, and refraining from treating the subject with a CACNA2D1 ligand, where antibodies to MBP84-104 are not detected in the subject.

Disclosed are kits for detecting antibodies to myelin basic protein-derived peptide (MBP84-104) where the kit includes (a) a solid support, where MBP84-104 is immobilized on the solid support; (b) a detection agent, where the detection agent comprises a detection element. In some forms, detection of the detection element can indicate the presence of the detection agent. In some forms, the presence of the detection agent can indicate the presence of an antibody to MBP84-104.

In some forms, the detection agent can be an anti-antibody antibody, where the anti-antibody antibody is an anti-IgM antibody or an anti-IgG antibody. In some forms, the kit can further comprise a reporter agent, where the reporter agent can facilitate detection of the detection element.

Also disclosed are kits for detecting antibodies to myelin basic protein-derived peptide (MBP84-104) where the kit includes (a) a solid support, where MBP84-104 is immobilized on the solid support; (b) a detection agent, where the detection agent comprises a detection element; and (c) a reporter agent, where the reporter agent can facilitate detection of the detection element. In some forms, detection of the detection element can indicate the presence of the detection agent. In some forms, the presence of the detection agent can indicate the presence of an antibody to MBP84-104.

Also disclosed are kits for detecting antibodies to myelin basic protein-derived peptide (MBP84-104) where the kit includes (a) a solid support, where MBP84-104 is immobilized on the solid support; (b) an anti-antibody antibody, where the anti-antibody antibody is an anti-IgM antibody or an anti-IgG antibody, where the anti-antibody antibody comprises a detection element. In some forms, detection of the detection element can indicate the presence of the anti-antibody antibody. In some forms, the presence of the anti-antibody antibody can indicate the presence of an antibody to MBP84-104.

Also disclosed are kits for detecting antibodies to myelin basic protein-derived peptide (MBP84-104) where the kit includes (a) a solid support, where MBP84-104 is immobilized on the solid support; (b) an anti-antibody antibody, where the anti-antibody antibody is an anti-IgM antibody or an anti-IgG antibody, where the anti-antibody antibody comprises a detection element; and (c) a reporter agent, where the reporter agent can facilitate detection of the detection element. In some forms, detection of the detection element can indicate the presence of the anti-antibody antibody. In some forms, the presence of the anti-antibody antibody can indicate the presence of an antibody to MBP84-104.

In some forms, the anti-antibody antibody, the reporter agent, and the detection element can be components of an enzyme-linked immunosorbent assay (ELISA) system. In some forms, the detection element can be an enzyme, where the enzyme catalyzes a reaction that can produce a detectable signal. In some forms, the reporter agent can be an enzymatic substrate for the enzyme, where the enzyme can act on the reporter agent to produce the detectable signal. In some forms, the solid support is in the form of a test strip. In some forms, the test strip is an immunochromatographic test strip.

Also disclosed are kits for detecting antibodies to myelin basic protein-derived peptide (MBP84-104) where the kit includes (a) one or more solid supports, where MBP84-104 is immobilized on at least one of the solid supports; (b) one or more antibodies, where at least one of the one or more antibodies is an antibody-detecting antibody, where each antibody-detecting antibody is independently an anti-IgM antibody or an anti-IgG antibody, where the antibody-detecting antibody comprises a detection element; and (c) a reporter agent, where the reporter agent can facilitate detection of the detection element. In some forms, detection of the detection element can indicate the presence of the anti-antibody antibody. In some forms, the presence of the anti-antibody antibody can indicate the presence of an antibody to MBP84-104.

Also disclosed are methods of detecting the existence of demyelination or neuropathic pain in a subject, where the method includes (a) bringing into contact a sample from the subject and disclosed solid support on which MBP84-104 is immobilized; (b) bringing into contact the solid support and the anti-antibody antibody; (c) bringing into contact the solid support and the reporter agent; and (d) detecting the presence of the reporter agent on the solid support. In some forms, the reporter agent produces a detectable signal. In some forms, detection of the detectable signal on the solid support indicates the presence of the reported agent on the solid support. In some forms, detection of the reporter agent indicates the presence of the detection element on the solid support. In some forms, detection of the detection element indicates the presence of the anti-antibody antibody on the solid support. In some forms, detection of the anti-antibody antibody indicates the presence of an antibody to MBP84-104 in the sample.

Also disclosed are methods of selecting a subject for treatment with a ligand for voltage-gated Ca²⁺-channel α2δ1 (CACNA2D1 ligand), where the method includes (a) bringing into contact a sample from the subject and the solid support of the kit of any one of claims 1-6 on which MBP84-104; (b) bringing into contact the solid support and the anti-antibody antibody; (c) bringing into contact the solid support and the reporter agent; (d) detecting the presence of the reporter agent on the solid support; and (e) selecting the subject for treatment with a composition consisting essentially of an effective amount of a CACNA2D1 ligand if an antibody to MBP84-104 is detected in the sample. Also disclosed are methods of selecting a subject for treatment with a ligand for voltage-gated Ca²⁺-channel α2δ1 (CACNA2D1 ligand), where the method includes (a) bringing into contact a sample from the subject and the solid support of the kit of any one of claims 1-6 on which MBP84-104; (b) bringing into contact the solid support and the anti-antibody antibody; (c) bringing into contact the solid support and the reporter agent; (d) detecting the presence of the reporter agent on the solid support; and (e) selecting the subject for treatment with a CACNA2D1 ligand if an antibody to MBP84-104 is detected in the sample. In some forms, the reporter agent produces a detectable signal. In some forms, detection of the detectable signal on the solid support indicates the presence of the reported agent on the solid support. In some forms, detection of the reporter agent indicates the presence of the detection element on the solid support. In some forms, detection of the detection element indicates the presence of the anti-antibody antibody on the solid support. In some forms, detection of the anti-antibody antibody indicates the presence of an antibody to MBP84-104 in the sample.

In some forms, the method can further comprise selecting the subject to not be treated with a composition consisting essentially of an effective amount of a CACNA2D1 ligand if an antibody to MBP84-104 is not detected in the sample. In some forms, the method can further comprise selecting the subject to not be treated with a CACNA2D1 ligand if an antibody to MBP84-104 is not detected in the sample.

In some forms, detection of an antibody to MBP84-104 in the sample indicates that the subject as has a disease or condition that causes, or is associated with, the presence or absence of, demyelination. In some forms, the disease or condition is neuropathic pain, including diabetic neuropathy, shingles, post herpetic neuralgia, neuromas, phantom limb pain and trigeminal neuralgia. In some forms, the disease or condition is an established or idiopathic chronic pain syndromes and conditions, including fibromyalgia and complex regional pain syndrome. In some forms, the disease of condition is demyelinating myelinoclastic disease or a demyelinating leukodystrophic disease. In some forms, the disease or condition is inflammatory demyelination, viral demyelination, acquired metabolic demyelination, hypoxic-ischemic demyelination, or compression-induced demyelination. In some forms, the disease or condition is diabetic neuropathy, shingles, post herpetic neuralgia, neuromas, phantom limb pain, trigeminal neuralgia, multiple sclerosis, acute multiple sclerosis, neuromyelitis optica, concentric sclerosis, acute-disseminated encephalonyelitis, acute hemorrhagic leucoencephalitis, progressive multifocal leucoencephalopathy, human immunodeficiency virus infection, subacute sclerosing panencephalitis, central pontine myelinlysis, extrapontine myelinolysis, fibromyalgia, or complex regional pain syndrome.

In some forms, the subject is suffering allodynia, dysesthesia, paraesthesia, lancinating, burning and other forms of pain. In some forms, the subject is female. In some forms, the sample is a serum sample.

In some forms, the method can further comprise administering a composition consisting essentially of an effective amount of a CACNA2D1 ligand to the subject if an antibody to MBP84-104 is detected in the sample. In some forms, the CACNA2D1 ligand can be gabapentin or pregabalin. In some forms, the method can further comprise refraining from administering a composition consisting essentially of an effective amount of a CACNA2D1 ligand to the subject if an antibody to MBP84-104 is not detected in the sample. In some forms, the method can further comprise administering a CACNA2D1 ligand to the subject if an antibody to MBP84-104 is detected in the sample. In some forms, the CACNA2D1 ligand can be gabapentin or pregabalin. In some forms, the method can further comprise refraining from administering a CACNA2D1 ligand to the subject if an antibody to MBP84-104 is not detected in the sample.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B describe an example of a peptide-based ELISA methodology using the highly conserved central algesic, immunodominant region 84-104 of MBP. FIG. 1A shows the sequence alignment of the evolutionary conserved central 84-104 region of MBP (MBP84-104) from human (Homo sapiens; SEQ ID NO:1), chimpanzee (Pan troglodytes; SEQ ID NO:2), pig (Sus scrofa; SEQ ID NO:3), guinea pig (Cavia porcellus; SEQ ID NO:4), rat (Rattus norvegicus; SEQ ID NO:5), mouse (Mus musculus; SEQ ID NO:6), cattle (Bos taurus; SEQ ID NO:7), rabbit (Oryctolagus cuniculus; SEQ ID NO:8), horse (Equus caballus; SEQ ID NO:9) and chicken (Gallus; SEQ ID NO:10). Conserved residues are in bold. Black rectangle, the algesic, immunodominant MBP84-104 epitope. Residues are numbered according to the human MBP sequence (GenBank # AAH08749). FIG. 1B is a schematic illustrating the ELISA methodology. The wells of a 96-well Maxisorp ELISA plate were coated with ExtrAvidin in bicarbonate buffer, pH 9.6. Following blocking with IgG- & protease-free BSA, the biotin-labeled MBP84-104 [wild-type (MBP84-104-WT) and scrambled control (MBP84-104-SCR)] peptides were each immobilized onto ExtrAvidin-coated wells. The serum samples were added to the wells. The capture antibodies were quantified using horseradish peroxidase (HRP)-conjugated species-specific IgG or IgM antibodies, and a HRP TMB/E substrate. The colorimetric reaction was stopped by acidification and the resulting A₄₅₀ was measured using a plate reader.

FIGS. 2A-2C are graphs showing that chronic constriction injury (CCI) of intact sciatic nerve induces sustained, unilateral mechanical allodynia in female rats concomitant with the upregulation of MMP-9 activity in injured nerve. FIG. 2A illustrates the results of von Frey behavioral testing in female rats at day 0 (prior to injury) and days 1, 3, 5, 7, and 28 post-CCI. A decline in the withdrawal threshold in the ipsilateral (IPSI) to CCI hind paw corresponds to allodynia. No sensitivity to stimuli below 10 grams was observed in the contralateral (CONTRA), uninjured hind paws. The mean withdrawal thresholds (gram force, g)±SEM of n=4-6/group. **, P<0.01. FIG. 2B shows the levels of MMP-9 and TIMP-1 mRNA in the sciatic nerve in female rats. Taqman qRT-PCR for MMP-9 (black) and TIMP-1 (grey) in sciatic nerve at days 0 (naïve) and days 1, 3, 5, 7, 14, and 28 post-CCI. The mean relative mRNA±SEM of n=4/group were normalized to glyceraldehyde 3-phosphate dehydrogenase, a housekeeping gene used for comparisons of gene expression data. To determine the fold-difference in the mRNA levels, the data are expressed as a percentage relative to day 1 (=100%). P<0.05. FIG. 2C shows the status of MMP-9 in sciatic nerve in female and male rats. Gelatin zymography analysis of sciatic nerve collected at day 28 post-CCI (representative of n=4/group) demonstrated a similar upregulation of MMP-9 in the CCI-injured nerve (CCI, ipsilateral to injury) relative to the intact control nerve (CTR, contralateral to injury) in both animal groups.

FIGS. 3A and 3B are graphs showing levels of urinary MMPs in a rat model of neuropathic pain. FIG. 3A shows the gelatinolytic urinary MMPs in females rats. The urine samples collected at day 0 (prior to injury) and days 1 and 28 post-CCI (n=4-6/group) were equilibrated in MMP buffer, pH 7.5 and then the protein concentrations were determined using the Bradford assay and made even by sample dilution in MMP buffer, pH 7.5. Dialyzed urine samples were analyzed by gelatin zymography. Gels were incubated in the absence and the presence of 20 mM EDTA (−EDTA and +EDTA, respectively), a strong chelator of metal ions and a broad spectrum MMP inhibitor. MMP-9, the latent MMP-9 control from HT1080 cells. NS, non-MMP activity band. FIG. 3B shows that, relative to the naïve animal, the specific MMP activity was secreted in the urine in a similar fashion in both CCI female and male rats (˜50 RFU/μg proteins), although the naïve males exhibited a higher background MMP activity. The urine samples collected at day 0 (CTR) and 28 post-CCI (CCI) (n=4-5/group) were equilibrated in MMP buffer, pH 7.5, and then the protein concentrations were determined using the Bradford assay. Dialyzed urine samples were co-incubated with the fluorescent Mca-PLGL-Dpa-AR-NH₂ MMP substrate in the presence and the absence of GM6001, a broad-spectrum hydroxamate MMP inhibitor. The specific MMP activity (RFU without GM6001-RFU with GM6001) is normalized to the protein concentrations. **, P<0.01.

Data are means±SE from multiple individual measurements performed in duplicate. RFU, relative fluorescence unit.

FIGS. 4A and 4B are graphs showing seropositivity for the algesic MBP peptide antibodies in female rats. FIG. 4A shows the results of an ELISA to assess the circulating anti-MBP84-104 peptide IgG and IgM antibodies in rat serum. The biotin-labeled MBP84-104-WT (diamond and triangle) and -SCR (square and cross) peptides were immobilized on the ExtrAvidin-coated wells of a 96-well plate. Serum aliquots collected at day 0 (prior to injury) and at days 7, 14, and 28 post-CCI were allowed to bind to the peptides. The bound antibodies were detected using HRP-conjugated anti-rat IgM and anti-rat IgG, and a TMB/E substrate. FIG. 4B shows the results of an ELISA of the IgG and IgM antibodies against intact, full-length MBP in rat serum. Intact, MBP (square and cross) and BSA (control; diamond and triangle) were immobilized in wells of a 96-well plate. Serum aliquots collected at day 0 (prior to injury) and at days 7, 14, and 28 post-CCI were allowed to bind to the wells. The bound antibodies were detected using HRP-conjugated anti-rat IgM and anti-rat IgG, and a TMB/E substrate. FIGS. 4A and 4B, data are means±SE from n=4/group and three individual experiments performed in triplicate.

FIGS. 5A and 5B are graphs showing that the upregulation of MMP-9 in CCI-injury is concomitant with seropositivity for the algesic MBP peptide antibodies in female, but not in male, rats. Both figures show the results of an ELISA of the anti-MBP84-104 peptide IgM antibodies in the serum from male and female rats (four animals/group, each). The biotin-labeled MBP84-104-WT and -SCR peptides were immobilized on the ExtrAvidin-coated wells of a 96-well plate. Serum aliquots collected from intact animals and at day 28 post-CCI were allowed to bind to the peptides. The bound antibodies were detected using HRP-conjugated anti-rat IgM and a TMB/E substrate for each serum sample. For simplicity, the combined data for four females and four males are shown. FIG. 5A depicts the specific A₄₅₀ values for the MBP84-104-WT peptide that are calculated relative to the MBP84-104-SCR peptide. FIG. 5B describes the fold-difference in the specific A₄₅₀ values for the MBP84-104-WT peptide between the intact (CTR) and CCI-injured (CCI) animals. **, P<0.01. FIGS. 5A and 5B, data are means±SE from at least three individual experiments performed in triplicate.

FIGS. 6A-6C are graphs displaying the seropositivity for the algesic MBP84-104 peptide antibodies in human female patients as determined by ELISA. FIGS. 6A and 6B show the results of an ELISA using serum samples from multiple sclerosis (MS) patients. The biotin-labeled MBP84-104-WT or -SCR peptides were immobilized on the ExtrAvidin-coated wells of a 96-well plate. Serum aliquots from two healthy volunteers (averaged values, CTR) and five MS patients (M-1 to M-5) were allowed to bind to the immobilized peptides. The bound IgG (black) and IgM (grey) antibodies were then detected using HRP-conjugated anti-human IgG or IgM, and a TMB/E substrate. FIG. 6A shows the specific A₄₅₀ values for the WT peptide that are calculated relative to the SCR peptide. FIG. 6B demonstrates the IgG- and IgM-fold difference in the specific A₄₅₀ values for the MBP84-104-WT peptide in MS patients relative to healthy volunteers (CTR=1). FIG. 6C shows the results of an ELISA using serum samples from fibromyalgia syndrome (FMS) patients. Serum samples from eight FMS patients (F-1 to F-8) were analyzed by ELISA with the immobilized MBP84-104-WT and -SCR peptides as described in FIGS. 6A and 6B. The average A₄₅₀ values for the serum of two healthy volunteers (CTR) and five MS patients (MS) were used for comparison purposes. The specific A₄₅₀ values for the WT peptide are calculated relative to the SCR peptide. Black and grey, the IgG and IgM levels against the MBP84-104-WT peptide, respectively. FIGS. 6A-6C, data are means±SE from three individual experiments performed in triplicate.

### DETAILED DESCRIPTION OF THE INVENTION

The disclosed method and compositions may be understood more readily by reference to the following detailed description of particular embodiments and the Example included therein and to the Figures and their previous and following description.

Disclosed are compositions, kits, and methods for detecting and assessing demyelinating diseases and conditions, neuropathic pain related to demyelinating diseases and conditions, selecting subjects for treatment with a ligand for voltage-gated Ca²⁺-channel α2δ1 (CACNA2D1 ligand), and selecting subjects to not be treated with a CACNA2D1 ligand. It has been discovered that the presence of antibodies to a proteolytic fragment of myelin basic protein (myelin basic protein-derived peptide (MBP84-104)) in subjects suffering from neuropathic pain indicates that (1) the subject is suffering from a demyelinating disease or condition and (2) that such subjects are more effectively treated with a CACNA2D1 ligand such as gabapentin or pregabalin as distinct from treatment with other pain relievers such as COX inhibitors (such as ketorolac), sodium channel blockers (such as lidocaine), NMDA antagonists (such as MK801), nonsteroidal anti-inflammatory drugs (NSAIDs) and opiates. These discoveries facilitate avoidance of unnecessary prescription of opioids, NSAIDS, and other problematic pain relievers in addition to the selection of an appropriate treatment.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a reporter agent is disclosed and discussed and a number of modifications that can be made to a number of molecules including the reporter agent are discussed, each and every combination and permutation of reporter agent and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B—F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Compounds and Compositions

Disclosed are compounds and compositions. Generally, these compounds and compositions are for use in the disclosed methods, such as the disclosed methods of treating subjects. The compounds and compositions are described positively, but any of the described compounds, compositions, and their combinations can be used to define compounds and compositions excluded from use, such as from use in the disclosed methods, such as the disclosed methods of treating subjects. Such inclusions, use, exclusions, and exclusion form use can be applied to any individual use or method, any set of group of uses or methods, or all of the methods or a class of methods.

Preferred compounds are ligands for voltage-gated Ca²⁺-channel α2δ1 (CACNA2D1 ligands). It has been discovered that such ligands affect voltage-gated Ca²⁺-channels that are involved in certain classes of neuropathic pain. In particular, it has been discovered that are effective in treating neuropathic pain in subjects having antibodies to a breakdown product of myelin: myelin basic protein-derived peptide (MBP84-104). It has been discovered that the presence of antibodies to MBP84-104 is indicative of certain disease conditions, such as demyelination, such as demyelinating myelinoclastic diseases and demyelinating leukodystrophic diseases. In contrast to many pain relievers, it has been discovered that CACNA2D1 ligands are effective for treating neuropathic pain that is associated with these disease conditions and with the presence of antibodies to MBP84-104.

In some forms, the compound is a CACNA2D1 ligand. In some forms, the CACNA2D1 ligand is gabapentin or pregabalin. In some forms, the CACNA2D1 ligand is gabapentin, pregabalin, gabapentin enacarbil, imagabalin, atagabalin, PD-217,014, 4-methylpregabalin, mirogabalin, phenibut, or baclofen. In some forms, the CACNA2D1 ligand is gabapentin, a specific antagonist of the α2δ1 subunit of voltage-gated calcium channels.

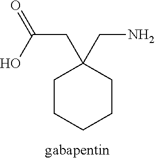 [SMILES: NCC1(CC(=O)O)CCCCC1]

Gabapentin is generally used to treat epilepsy, neuropathic pain, hot flashes, and restless leg syndrome (Spencer et al. J. Neurosci 34(25):8605-8611 (2014); Wijemanne; Sleep Medicine 16(6):678-690 (2015)).

In some forms, the CACNA2D1 ligand is gabapentin enacarbil, a prodrug to gabapentin. Gabapentin enacarbil was designed for increased oral bioavailability over gabapentin, and is indicated for the treatment of restless legs syndrome (Cundy et al, J Pharmacol Exp Ther 311(1):315-323 (2004); Cundy et al, J Pharmacol Exp Ther 311(1):324-333 (2004); Imamura and Kushida (Expert Opin Pharmacotherapy 11(11):1925-1932 (2010)).

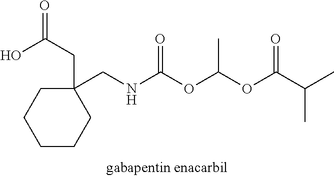 [SMILES: CC(OC(=O)NCC1(CC(=O)O)CCCCC1)OC(=O)C(C)C]

In some forms, the CACNA2D1 ligand is pregabalin, another antagonist of the α2δ1 subunit of voltage-gated calcium channels. Pregabalin is generally used to treat epilepsy, neuropathic pain, fibromyalgia, and generalized anxiety disorder (Frampton CNS Drugs 28(9):835-854 (2014); Patel and Dickenson Pharm Res Perspect 4(2):e00205 (2016)).

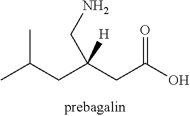

In some forms, the CACNA2D1 ligand is imagabalin, a specific antagonist of the α2δ1 subunit of voltage-gated calcium channels. Imagabalin has demonstrated efficacy for anxiolytic, analgesic, hypnotic, and anticonvulsant-like activity, as well as for generalized anxiety disorder (Ereshefsky; 2008 Press Release; A 10-Week Study Evaluating the Efficacy And Safety of PD 0332334 for the Treatment of Generalized Anxiety Disorder; ClinicalTrials.gov NCT00542685).

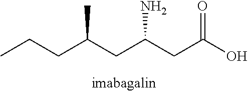

In some forms, the CACNA2D1 ligand is atagabalin, a drug related to gabapentin, which similarly binds to the α2δ1 subunit of the voltage-gated Ca²⁺ channel, and is indicated for treatment of insomnia (Corrigan et al. Brit J Clin Pharmacol 68(2):174-180 (2009); Kjellsson et al. Pharmaceut Res 28(10)2610-2627 (2011)).

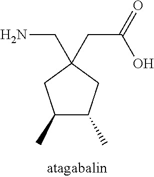

In some forms, the CACNA2D1 ligand is PD-217,014. PD-217,014 is related to gabapentin, and is similarly an antagonist of the α2δ1 subunit of the voltage-gated Ca²⁺ channel PD-217,014 produces visceral analgesic effects in animal studies with higher potency and efficacy than gabapentin (Ohashi et al. Pharmacology 81(2):144-150 (2008)).

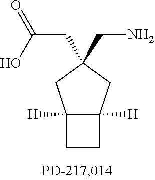 [SMILES: NC[C@@]1(CC(=O)O)C[C@H]2CC[C@H]2C1]

In some forms, the CACNA2D1 ligand is 4-methylpregabalin. 4-methylpregabalin acts as an analgesic with effectiveness against difficult to treat “atypical” pain syndromes such as neuropathic pain (Belliotti et al. J Med Chem 48(7):2294-2307 (2005)). This drug typically finds use as an anticonvulsant, muscle relaxant, anxiolytic, and mood stabilizer.

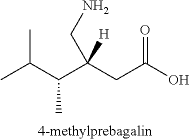

In some forms, the CACNA2D1 ligand is mirogabalin, a drug that is related to gabapentin and pregabalin and is also an antagonist of the α2δ1 subunit of the voltage-gated Ca²⁺ channel Mirogabalin is indicated for treatment of diabetic peripheral neuropathic pain (Vinik et al. Diabetes Care 37(12):3253-3253 (2014); Vinik et al. Neurology 82(10):S20.004 (2014)).

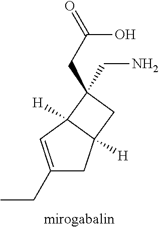

In some forms, the CACNA2D1 ligand is the antagonist phenibut, which is generally used for its anxiolytic effects (Lapin CNS Drug Rev 7(4)471-481 (2001)).

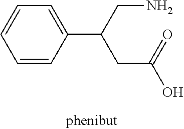 [SMILES: NCC(CC(=O)O)c1ccccc1]

In some forms, the CACNA2D1 ligand is the antagonist baclofen.

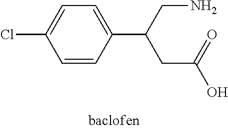 [SMILES: NCC(CC(=O)O)c1ccc(Cl)cc1]

Baclofen serves as a central nervous system depressant and skeletal muscle relaxant, and is indicated in pain management (Cherny et al. Oxford Textbook of Palliative Medicine; Oxford University Press p. 585 (2015)).

In some forms, the CACNA2D1 ligand is w-Agatoxin IVA, a peptide originally isolated from funnel web-spider venom Agelenopsis aperta. This peptide specifically blocks the α2δ1 subunit of voltage-gated calcium channels (Adams Toxicon 43(5):509-525 (2004)).

In some forms, the CACNA2D1 ligand is a conotoxin. Conotoxins are peptides consisting of 10 to 30 amino acid residues, typically having one or more disulfide bonds. ω-conotoxin, in particular, blocks the α2δ1 subunit of voltage-gated calcium channels (Needham et al. Neurogastroenterol Motil 22(10):e301-308 (2010)).

In some forms, the CACNA2D1 ligand is NVA1309, an agonist of the α2δ1 subunit of the voltage-gated Ca²⁺ channel. NVA1309 has nanomolar affinity for its target and does not penetrate the brain (Hesselink; J. Pharm Clin Res 1(5):555575 (2016)).

In some forms, the ligand is comprised in a composition. In some forms, the composition does not include an opioid or a non-steroidal anti-inflammatory drug (NSAID). In some forms, the composition further comprises one or more pain relievers.

Examples of pain relievers include Abenol, Acephen, Aceta, Aceta-Gesic, acetaminophen, aspirin, dihydrocodeine, phenyltoloxamine, salicylamide, codeine, dextromethorphan, doxylamine, diphenhydramine, guaifenesin, hydrocodone, oxycodone, phenyltoloxamine, tramadol, Actamin, Actimol Children's, Actimol Infant, Actiprofen, Actiq, Acuflex, Addaprin, Advil, Aflaxen, A-G Profen, Aleve, Aleve PM, Alfenta, alfentanil, Ali-Flex, Alka-Seltzer Wake-Up Call!, All Day Pain Relief, All Day Relief, Aloe Vera Burn Relief Spray with Lidocaine, Altenol, Aminofen, amitriptyline, Anacin, Anacin Aspirin Free, Anaprox, Anaprox-DS, Anbesol, AneCream, AneCream with Tegaderm, Anestacon, Anestafoam, Anexsia, Ansaid, Apicaine-X, Apra, Arctic Relief, Arthritis Pain, Arthritis Pain Relief, Arymo ER, Ascriptin, Aspercreme, Aspergum, aspirin, butalbital, carisoprodol, meprobamate, Aspiritab, Aspirtab, Astero, Astramorph PF, Atasol, Axsain, Bactine, Bayer Aspirin, Bayer Back & Body, BC Arthritis, BC Fast Pain Relief, Be-Flex Plus, Belbuca, benzocaine, benzocaine/dextromethorphan, Benzo-Jel, Berri-Freez, Blistex Pro Relief, Buffasal, Bufferin Low Dose, bupivacaine liposome, Buprenex, buprenorphine, Butalbital Compound, butorphanol, Butrans, Cafgesic, Caldolor, camphor, Capital w/ Codeine, capsaicin, capsaicin, diclofenac, lidocaine, Capsicum Oleoresin, Capsin, Capzasin, Capzasin Back and Body, Capzasin-HP, Capzasin-P, Castiva Warming, Cataflam, Catapres, Celebrex, celecoxib, Cepacol Ultra, Cetafen, Chiggerex, Choline Magnesium Trisalicylate, choline salicylate/magnesium salicylate, CidalEaze, Clear Cough PM Multi-Symptom, Clinoril, clonidine, Cocet Plus, codeine, Codrix, Co-Gesic, Cold Spot Point Relief, Comtrex Deep Chest Cold, Contac Cold+Flu (Night) Cooling Relief Liquid, ConZip, Cope, Coricidin HBP Nighttime Multi-Symptom Cold, Curasore, Dazidox, Demerol, Dendracin Neurodendraxcin, Denti-Care Denti-Freeze, Dent-O-Kain, Derma Numb, DermacinRx Lexitral PharmaPak, DermacinRx Lido V Pak, DermacinRx Lidotral, Derma-Pax, Dermarest, Dermoplast, DeWitt's Pain Reliever, diclofenac, Diclofex DC, diflunisal, Dilaudid, Dilaudid-HP, diphenhydramine, ibuprofen, magnesium salicylate, naproxen, Doan's Pills, Doans PM, Dolacet, Dolagesic, Dolobid, Dologesic, Dologesic DF, Dolono, Dolophine, Dolorex, duloxetine, Durabac, Duraclon, Duragesic, Duramorph, Duraxin, dyclonine, Dyloject, Easprin, EC-Naprosyn, Ecotrin, Ecpirin, Eha Lotion, Elixsure Fever/Pain, Embeda, Endocet, Endodan, Entercote, Equagesic, Equaline Pain Relief, ETH-Oxydose, etodolac, Exalgo, Excedrin PM, Excedrin Quick Tab, Excedrin Tension Headache, Exparel, Farbital, Fasprin, Fast Freeze, Febrol Solution, Feldene, fenoprofen, Fenortho, fentanyl, Fentora, Feverall, Fiorinal, Flanax Pain Reliever, Flector Patch, flurbiprofen, Freeze It, gabapentin, Gebauer's Spray and Stretch, Genacote, Genapap, Genebs, Genpril, Glydo, GNP Capsaicin, Goody's Body Pain, Halfprin, Haltran, Headache Relief PM, Hycet, Hydrocet, hydrocodone, hydromorphone, hydroxyzine, Hysingla ER, IBU, IBU-200, Ibu-4, Ibu-6, Ibu-8, Ibudone, ibuprofen, ibuprofen/oxycodone, Ibuprofen PM, Ibu-Tab, Icy Hot PM Lotion, Icy Hot PM Patch, imipramine, Indocin, indomethacin, Infant's Tylenol, Infumorph, Ionsys, Jr. Tylenol, Kadian, Kank-a, ketamine, ketoprofen, ketorolac, Klofensaid II, Lagesic, Lanacane, Laryngesic, Laryng-O-Jet Spray, Legatrin PM, Levacet, Levo-Dromoran, levorphanol, LidaMantle, lidocaine, Lidocaine Viscous, Lidocream, Lidopac, Lidopin, LidoRx, LidoRxKit, Lidosense 5, Lidotrans 5 Pak, Lidovex, Lidozol, Liquicet, LMX 4, LMX 5, Lodine, Lorcet, Lortab, LTA II Kit, Magnacet, magnesium salicylate, Mapap, Maxidone, meclofenamate, Medicone, Medi-Derm Rx, Medi-Quik Spray, Medi-Seltzer, Medi-Tabs, Medrox, Medrox-Rx, mefenamic acid, Menthac Arthritis Cream with Capsaicin, Menthocin Patch with Lidocaine, menthol, meperidine, promethazine, Meperitab, methadone, Methadone Diskets, Methadose, Micrainin, Midol Extended Relief, Midol IB, Midol PM, MorphaBond, morphine, naltrexone, Motrin, MS Contin, MST, Myoflex Cream, Myophen, nalbuphine, Nalfon, naloxone, pentazocine, naltrexone, Naprelan, Naprosyn, naproxen, Narvox, Night Time Pain, Norco, Nortemp Children's, nortriptyline, Norwich Aspirin, Nucynta, Nucynta ER, NuDiclo SoluPak, Numorphan, Nuprin, Nuprin Backache Caplet, Ofirmev, Opana, Orabase, Oramorph SR, Orudis, Orudis KT, Oruvail, Outgro Pain Relief, Oxaydo, oxycodone, OxyContin, Oxydose, Oxyfast, OxyIR, oxymorphone, P-A-C, P-A-C Analgesic, Pain Relief PM Extra Strength, Painaid, Palladone, Panlor DC, Panlor SS, Paracetamol, pentafluoropropane, tetrafluoroethane, pentazocine, Percocet, Percodan, Percogesic, Percogesic Extra Strength, Perform Pain Relieving Spray, Perloxx, piroxicam, Ponstel, Pramox, pramoxine, Prax, Prax Wipe, Precaine B, pregabalin, Prialt, Primlev, Proctofoam, Proprinal, Q-Pap, Q-Profen, Qutenza, RadiaGuard, RectiCare, Redutemp, Regenecare HA Spray, Relagesic, Rematex, Renovo LidoS, Reprexain, RhinoFlex 650, RMS, Robitussin Peak Cold Nighttime Cold+Flu, Roxanol, Roxicet, Roxicodone, Roxicodone Intensol, Roxilox, RoxyBond, Rybix ODT, Ryzolt, Salonpas Gel-Patch, Salonpas Pain Patch with Capsaicin, Sarna Sensitive, Sarna Ultra, Senatec, Silapap Childrens, Silvera Pain Relief, Sloan's Liniment, Solarcaine First Aid Medicated Spray, Soma Compound with Codeine, Soothee Patch, Soothing Liniment, Sprix, Stagesic, Stanback, Stanback Fast Pain Relief, Sting Relief, Sting-Kill, Stopain, Sublimaze, sulindac, Super Dent Topical Anesthetic Gel, Sure Result DSS Premium Pak, Synalgos-DC, Tactinal, Talwin, tapentadol, Tempra Quicklets, Tetramex, Theracodophen Low 90, Theraflu Flu & Chest Congestion, Tiger Balm, Tivorbex, Tofranil, Tolectin, tolmetin, Topcare 8 Hour Pain Relief, Topcare Arthritis Pain Relief, Topcare Cough and Sore Throat, Topicaine, Topical Anesthetic Dental Gel, Toradol, tramadol, Tramapap, Tranzarel, Trezix, Triaminic Cough & Sore Throat, Triaminic Softchews Cough & Sore Throat, Tricosal, Trilisate, Trixaicin, Trocaine, trolamine salicylate, Tronolane, Troxyca ER, Tycolene, Tylenol, Tylenol with Codeine, Tylophen, Tylox, Ultracet, Ultram, Unisom PM Pain, Vantrela ER, Verdrocet, Vicks NyQuil Cold & Flu Nighttime Relief, Vicodin, Vicoprofen, Vistaril, Vitapap, Voltaren, Xartemis XR, Xodol, Xolox, Xrylix, Xtampza ER, Xylocaine Jelly, Xylocaine Topical, Xylocaine Viscous, Xylon 10, Zamicet, Zerlor, Zflex, Zgesic, ziconotide, Zipsor, Zohydro ER, Zolvit, ZORprin, Zorvolex, Zostrix, Zydone, and Zyfrel. Any of these pain relievers, and any sets or subgroups of these pain relievers, can be specifically included or excluded in or from a composition or in or from use in a method.

B. Solid Supports

Solid supports are used to hold or immobilize the disclosed proteins, peptides, antigens, antibodies, and other components. Solid supports are solid-state substrates or supports with which molecules (such as peptides and proteins) or other components used in, or produced by, the disclosed methods can be associated. Molecules can be associated with solid supports directly or indirectly. For example, peptides can be bound to the surface of a solid support. An array is a solid support to which multiple peptides or other molecules have been associated in an array, grid, or other organized pattern.

Solid-state substrates for use in solid supports can include any solid material with which components can be associated, directly or indirectly. This includes materials such as acrylamide, agarose, carboxylated poly(vinyl chloride) (CPVC), cellulose acetate membrane, cellulose nitrate (CN) membrane, cellulose, collagen, filter paper (Whatman), fluorocarbons, functionalized silane, Glass fiber filters (GFC) (A,B,C), glass, glycosaminoglycans, gold, latex, mixed cellulose ester membrane, nitrocellulose, nylon, plastic, polyamino acids, polyanhydrides, polycarbonates, polyethersulfone (PES) membrane, polyethylene oxide, polyethylene vinyl acetate, polyethylene, polyethylimine coated GFCs, polyglycolic acid, polylactic acid, polymethacrylate, polyorthoesters, polypropylene, polypropylfumerate, polysilicates, polystyrene, polyvinylidene fluoride (PVDF), porous mylar or other transparent porous films, PTFE membrane, silicon rubber, teflon, and ultrafiltration membranes of poly(vinyl chloride) (PVC). Solid-state substrates can have any useful form including beads, bottles, chemically-modified glass slides, column matrix, cross-linked polymer beads, dishes, fibers, mass spectrometer plates, membranes, microparticles, microtiter dishes, particles, shaped polymers, slides, sticks, test strips, thin films, thin membranes, and woven fibers, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of molecules, compounds or peptides immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of molecules, compounds and/or peptides can be distributed over any number of solid supports. For example, at one extreme, each component can be immobilized in a separate reaction tube or container, or on separate beads or microparticles.

Methods for immobilization of proteins and peptides to solid-state substrates are well established.

Each of the components immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

C. Detection Agents

A detection agent is a specific binding molecule that also comprises or is coupled to a detection element. The specific binding molecule can be referred to as the affinity portion of the detection agent and the detection element is referred to as the detection element portion of the detection agent. As used herein, a specific binding molecule is a molecule that interacts specifically with a particular molecule or moiety. The molecule or moiety that interacts specifically with a specific binding molecule is referred to herein as a target molecule. An anti-MBP84-104 antibody, an IgG antibody, and an IgM antibody are examples of target molecules. It is to be understood that the term target molecule refers to both separate molecules and to portions of molecules, such as an epitope of a protein, that interacts specifically with a specific binding molecule. For example, the IgG or IgM determinant of an antibody can be the portion of an antibody that a specific binding molecule interacts with. Antigens, antibodies, either member of a receptor/ligand pair, and other molecules with specific binding affinities are examples of specific binding molecules, useful as the affinity portion of a detection agent. A detection agent with an affinity portion that is an antibody can be referred to herein as a detection antibody. By coupling a detection element to such specific binding molecules, binding of a specific binding molecule to its specific target can be detected by detecting the detection element. A detection agent that interacts specifically with a particular target molecule is said to be specific for that target molecule. For example, a detection agent with an affinity portion which is an antibody that binds to a particular antigen is said to be specific for that antigen. The antigen is the target molecule. Detection agents are also referred to herein as detection molecules.

A preferred form of detection agent is an anti-antibody antibody. An anti-antibody is an antibody that is specific for a particular antibody or class of antibodies. As useful form of anti-antibody antibodies is antibodies specific for antibody class determinants or, put another way, specific for antibodies of a particular class (such as IgG and IgM antibody classes). Because the antibody class determinants are often species-specific, it is possible and useful to us anti-antibody antibodies that are specific to antibodies from a particular species. Anti-antibody antibodies that are specific for human IgG antibodies or human IgM antibodies, for example, are useful for binding to and aiding in detection of human antibodies.

D. Detection Elements

To aid in detection of anti-MBP84-104 antibodies, detection elements can be directly can be associated with or coupled to detection agents. As used herein, a detection element is any molecule that can be associated with amplified nucleic acid, directly or indirectly, and which results in a measurable, detectable signal, either directly or indirectly. Many such labels for are known to those of skill in the art. Examples of suitable detection elements include radioactive isotopes, fluorescent molecules, phosphorescent molecules, enzymes, antibodies, and ligands.

The disclosed detection elements can be part of, and detectable with, enzyme-linked detection systems. Enzyme-linked detection generally involves an enzyme as a label or tag on a component where the presence of the enzyme (and thus of the analyte with which the enzyme is associated) is detected by having the enzyme convert an enzymatic substrate into a form that produces a detectable signal. For example, analytes labeled or associated with alkaline phosphatase can be detected by adding the chemiluminescent substrate CSPD (Tropix, Inc.). The fluorescent reaction product can then be detected. Preferred forms of detection elements are enzymes, such as alkaline phosphatases and peroxidases, for use in an enzyme-linked detection system.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine) Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Detection elements such as biotin can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.).

Molecules that combine two or more of these detection elements are also considered detection elements. Any of the known detection elements can be used with the disclosed detection agents. Methods for detecting and measuring signals generated by detection elements are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection element coupled to the antibody. Such methods can be used directly in the disclosed method of amplification and detection. As used herein, detection agents are molecules which interact with amplified nucleic acid and to which one or more detection elements are coupled.

E. Reporter Agents

Reporter agents are molecules, compounds, or components that can facilitate detection of detection elements. Reporter agents are most useful when the detection element does not produce a detectable signal or a conveniently detectable signal. In some forms, the reporter agent can generate or be converted into a detectable signal or as molecule, compound, or component that produces a detectable signal. For example, if the detection element is an enzyme, the reporter agent can be a substrate for the enzyme here the enzymatic product of the reporter agent is or produces a detectable signal. In some forms, the reporter agent can be or comprise a detectable signal. In these forms, association of the reporter agent with the detection element associates the detectable signal with the detection agent. This essentially labels the detection agent with the detectable signal of the reported agent.

Preferred reporter agents are enzymatic substrates, such as substrates that produce a detectable signal upon reaction with their respective enzyme. Such reporter agents are thus part of an enzyme-linked detection system, with the enzyme associated with or coupled to a detection agent (with the enzyme thus serving as a detection element).

F. Kits

Disclosed are kits for detecting antibodies to myelin basic protein-derived peptide (MBP84-104). The materials described herein as well as other materials can be packaged together in any suitable combination as a kit useful for performing, or aiding in the performance of, the disclosed method. It is useful if the kit components in a given kit are designed and adapted for use together in the disclosed method. For example, disclosed are kits that include (a) a solid support, where MBP84-104 is immobilized on the solid support; (b) a detection agent, wherein the detection agent comprises a detection element. In some forms, detection of the detection element can indicate the presence of the anti-antibody. In some forms, the presence of the anti-antibody antibody can indicate the presence of an antibody to MBP84-104.

In some forms, the kit can include (a) a solid support, where MBP84-104 is immobilized on the solid support; (b) an anti-antibody antibody, where the anti-antibody antibody is an anti-IgM antibody or an anti-IgG antibody, where the anti-antibody antibody comprises a detection element; and (c) a reporter agent, where the reporter agent can facilitate detection of the detection element. In some forms, the anti-antibody antibody, the reporter agent, and the detection element can be components of an enzyme-linked immunosorbent assay (ELISA) system. In some forms, the detection element can be an enzyme, where the enzyme catalyzes a reaction that can produce a detectable signal. In some forms, the reporter agent can be an enzymatic solid support for the enzyme, where the enzyme can act on the reporter agent to produce the detectable signal. In some forms, the solid support is in the form of a test strip. In some forms, the test strip is an immunochromatographic test strip.

In some forms, the kit can include (a) one or more solid supports, where MBP84-104 is immobilized on at least one of the solid supports; (b) one or more antibodies, where at least one of the one or more antibodies is an antibody-detecting antibody, where each antibody-detecting antibody is independently an anti-IgM antibody or an anti-IgG antibody, where the antibody-detecting antibody comprises a detection element; and (c) a reporter agent, where the reporter agent can facilitate detection of the detection element. In some forms, detection of the detection element can indicate the presence of the anti-antibody antibody. In some forms, the presence of the anti-antibody antibody can indicate the presence of an antibody to MBP84-104.

G. Mixtures

Disclosed are mixtures formed by performing or preparing to perform the disclosed method. For example, disclosed are mixtures comprising a solid support and a detection agent; a solid support, a detection agent, and a reporter agent; a sample and a solid support; a sample, a solid support, and a detection agent; a sample, a solid support, a detection agent, and a reporter agent; a solid support and an anti-antibody antibody; a solid support, an anti-antibody antibody, and a reporter agent; a sample, a solid support, and an anti-antibody antibody; a sample, a solid support, an anti-antibody antibody, and a reporter agent; a test strip and a detection agent; a test strip, a detection agent, and a reporter agent; a sample and a test strip; a sample, a test strip, and a detection agent; a sample, a test strip, a detection agent, and a reporter agent; a multi-well plate and a detection agent; a multi-well plate, a detection agent, and a reporter agent; a sample and a multi-well plate; a sample, a multi-well plate, and a detection agent; a sample, a multi-well plate, a detection agent, and a reporter agent; a solid support, a detection agent, and an enzymatic substrate; and a sample, a solid support, a detection agent, and an enzymatic substrate.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

H. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising a disclosed kit and an apparatus for detecting a detectable signal; a disclosed kit, an apparatus for processing samples and components of the kit according to one or more of the disclosed methods, and an apparatus for detecting a detectable signal; and a disclosed kit and an apparatus for (a) processing samples and components of the kit according to one or more of the disclosed methods and (b) detecting a detectable signal.

I. Data Structures and Computer Control

Disclosed are data structures used in, generated by, or generated from, the disclosed method. Data structures generally are any form of data, information, and/or objects collected, organized, stored, and/or embodied in a composition or medium. Results of the disclosed method stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods, kits, and compositions are applicable to numerous areas including, but not limited to, selecting subjects for treatment with a CACNA2D1 ligand such as gabapentin or pregabalin, selecting subjects to not be treated with a CACNA2D1 ligand such as gabapentin or pregabalin, selecting subjects to not be treated with pain relievers such as COX inhibitors (such as ketorolac), sodium channel blockers (such as lidocaine), and NMDA antagonists (such as MK801). Other uses are disclosed, apparent from the disclosure, and/or will be understood by those in the art.

A. Actions Based on Identifications

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, the disclosed compositions, kits, and methods are useful for detecting and assessing demyelinating diseases and conditions, neuropathic pain related to demyelinating diseases and conditions, selecting subjects for treatment with a ligand for voltage-gated Ca²⁺-channel α2δ1 (CACNA2D1 ligand), and selecting subjects to not be treated with a CACNA2D1 ligand. Such detections, assessments, and selections are forms of identifications. It has been discovered that the presence of antibodies to a proteolytic fragment of myelin basic protein (myelin basic protein-derived peptide (MBP84-104)) in subjects suffering from neuropathic pain indicates that (1) the subject is suffering from a demyelinating disease or condition and (2) that such subjects are more effectively treated with a CACNA2D1 ligand such as gabapentin or pregabalin as distinct from treatment with other pain relievers such as COX inhibitors (such as ketorolac), sodium channel blockers (such as lidocaine), and NMDA antagonists (such as MK801). Thus, for example, detection of anti-MBP84-104 antibodies in a sample from a subject identifies the subject as suffering from a demyelinating disease or condition. Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods comprising making one or more further identifications based on one or more other identifications. For example, particular treatments, monitorings, follow-ups, advice, etc. can be identified based on the other identification. For example, identification of a subject as having a disease or condition with a high level of a particular component or characteristic can be further identified as a subject that could or should be treated with a therapy based on or directed to the high level component or characteristic. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

As another example, disclosed are methods comprising treating, monitoring, following-up with, advising, etc. a subject identified in any of the disclosed methods. Also disclosed are methods comprising treating, monitoring, following-up with, advising, etc. a subject for which a record of an identification from any of the disclosed methods has been made. For example, particular treatments, monitorings, follow-ups, advice, etc. can be used based on an identification and/or based on a record of an identification. For example, a subject identified as having a disease or condition with a high level of a particular component or characteristic (and/or a subject for which a record has been made of such an identification) can be treated with a therapy based on or directed to the high level component or characteristic. Such treatments, monitorings, follow-ups, advice, etc. can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

The disclosed measurements, detections, comparisons, analyses, assays, screenings, etc. can be used in other ways and for other purposes than those disclosed. Thus, the disclosed measurements, detections, comparisons, analyses, assays, screenings, etc. do not encompass all uses of such measurements, detections, comparisons, analyses, assays, screenings, etc.

### Methods

Disclosed are methods of detecting anti-MBP84-104 antibodies. Also disclosed are methods of detecting the existence of demyelination or neuropathic pain in a subject. Also disclosed are methods of treating subjects that have anti-MBP84-104 antibodies. Also disclosed are methods of treating subjects that have demyelination or neuropathic pain. Also disclosed are methods of treating subjects that have a demyelinating disease or condition. Also disclosed are methods of treating subjects for pain associated with MBP84-104 antibodies. In preferred forms, each of these methods can involve detecting anti-MBP84-104 antibodies. It has been discovered that the presence of anti-MBP84-104 antibodies in a subject is indicative of demyelination or neuropathic pain in the subject, the presence of demyelinating disease or condition in the subject, and/or neuropathic pain in the subject that is amenable to treatment with a ligand for voltage-gated Ca²⁺-channel α2δ1 (CACNA2D1 ligand).

In some forms, the method can include (a) bringing into contact a sample from the subject and disclosed solid support on which MBP84-104 is immobilized; (b) bringing into contact the solid support and the anti-antibody antibody; (c) bringing into contact the solid support and the reporter agent; and (d) detecting the presence of the reporter agent on the solid support. In some forms, the reporter agent produces a detectable signal. In some forms, detection of the detectable signal on the solid support indicates the presence of the reported agent on the solid support. In some forms, detection of the reporter agent indicates the presence of the detection element on the solid support. In some forms, detection of the detection element indicates the presence of the anti-antibody antibody on the solid support. In some forms, detection of the anti-antibody indicates the presence of an antibody to MBP84-104 in the sample.

In some forms, the method can include (a) bringing into contact a sample from the subject and the solid support of the kit of any one of claims 1-6 on which MBP84-104; (b) bringing into contact the solid support and the anti-antibody antibody; (c) bringing into contact the solid support and the reporter agent; (d) detecting the presence of the reporter agent on the solid support; and (e) selecting the subject for treatment with a CACNA2D1 ligand if an antibody to MBP84-104 is detected in the sample. In some forms, the reporter agent produces a detectable signal. In some forms, detection of the detectable signal on the solid support indicates the presence of the reported agent on the solid support. In some forms, detection of the reporter agent indicates the presence of the detection element on the solid support. In some forms, detection of the detection element indicates the presence of the anti-antibody antibody on the solid support. In some forms, detection of the anti-antibody antibody indicates the presence of an antibody to MBP84-104 in the sample.

In some forms, the method can further comprise selecting the subject to not be treated with a CACNA2D1 ligand if an antibody to MBP84-104 is not detected in the sample.

In some forms, detection of an antibody to MBP84-104 in the sample indicates that the subject as has a disease or condition that causes, or is associated with, the presence of, demyelination or neuropathic pain. In some forms, the disease or condition is a demyelinating myelinoclastic disease or a demyelinating leukodystrophic disease. In some forms, the disease or condition is inflammatory demyelination, viral demyelination, acquired metabolic demyelination, hypoxic-ischemic demyelination, or compression-induced demyelination. In some forms, the disease or condition is diabetic neuropathy, shingles, post herpetic neuralgia, neuromas, phantom limb pain, trigeminal neuralgia, multiple sclerosis, acute multiple sclerosis, neuromyelitis optica, concentric sclerosis, acute-disseminated encephalonyelitis, acute hemorrhagic leucoencephalitis, progressive multifocal leucoencephalopathy, human immunodeficiency virus infection, subacute sclerosing panencephalitis, central pontine myelinlysis, extrapontine myelinolysis, fibromyalgia, or complex regional pain syndrome.

In some forms, the subject is suffering allodynia. In some forms, the subject is female. In some forms, the sample is a serum sample.

In some forms, the method can further comprise administering a ligand for voltage-gated Ca²⁺-channel α2δ1 (CACNA2D1 ligand) to the subject if an antibody to MBP84-104 is detected in the sample. In some forms, the CACNA2D1 ligand can be gabapentin or pregabalin. In some forms, the method can further comprise refraining from administering a ligand for voltage-gated Ca²⁺-channel α2δ1 (CACNA2D1 ligand) to the subject if an antibody to MBP84-104 is not detected in the sample.

A demyelinating disease is any disease of the nervous system in which the myelin sheath of neurons is damaged. This damage impairs the conduction of signals in the affected nerves. In turn, the reduction in conduction ability causes deficiency in sensation, movement, cognition, or other functions depending on which nerves are involved.

Some demyelinating diseases are caused by genetics, some by infectious agents, some by autoimmune reactions, and some by unknown factors. Organophosphates, a class of chemicals which are the active ingredients in commercial insecticides such as sheep dip, weed-killers, and flea treatment preparations for pets, etc., will also demyelinate nerves. Neuroleptics can also cause demyelination (Konopaske et al., Biol. Psychiatry. 63 (8): 759-65 (2008)).

Demyelinating diseases are traditionally classified in two kinds: demyelinating myelinoclastic diseases and demyelinating leukodystrophic diseases. In the first group a normal and healthy myelin is destroyed by a toxic, chemical or autoimmune substance. In the second group, myelin is abnormal and degenerates (Fernandez et al., Medicine. 11 (77): 4601-4609 (2015)). The second group has also be referred to as dysmyelinating diseases (Poser, Arch Neurol. 4 (3): 323-332 (1961)).

In the most known example, multiple sclerosis, there is good evidence that the body's own immune system is at least partially responsible. Acquired immune system cells called T-cells are known to be present at the site of lesions. Other immune system cells called Macrophages (and possibly Mast cells as well) also contribute to the damage. Vitamin B12 deficiency can cause demyelination

A. Antibody-Based Assays

An immunoassay, and its attendant materials, can be used to detect the presence of antibodies specific to MBP84-104 in a sample. An immunoassay is an assay that uses an antibody to specifically bind an antigen (e.g., a biomarker). An immunoassay is characterized by the use of specific binding properties of a particular antibody to isolate, target, and/or quantify the antigen or, as is the case with the disclosed methods, use of a specific antigen to isolate, target, and/or quantify an antibody to the antigen based on specific binding properties of the antibody. Thus, under designated immunoassay conditions, the particular protein or peptide binds to specified antibodies at least two times the background and do not substantially bind in a significant amount to other proteins present in the sample. Specific binding of a protein or peptide to an antibody under such conditions generally depends on a specific protein, peptide, or other antigen for binding by an antibody having specificity for the specific protein, peptide, or other antigen. In the case of MBP84-104, being from a self-protein, antibodies to it would not normally occur in subjects absent a disease condition.

Generally, a sample obtained from a subject can be contacted with the protein, peptide, or other antigen that is specifically bound by the antibody. Optionally, the protein, peptide, or other antigen can be fixed to (immobilized on) a solid support to facilitate washing and subsequent isolation of the complex, prior to contacting the protein, peptide, or other antigen with a sample. Examples of solid supports include glass or plastic in the form of, e.g., a microtiter plate, a stick, a bead, or a microbead.

Methods for measuring the amount or presence of an antibody-biomarker complex include, for example, detection of fluorescence, luminescence, chemiluminescence, absorbance, reflectance, transmittance, birefringence or refractive index (e.g., surface plasmon resonance, ellipsometry, a resonant mirror method, a gating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Useful assays are well known in the art, including, for example, an enzyme immune assay (EIA) such as enzyme-linked immunosorbent assay (ELISA), a radioimmune assay (RIA), immunoprecipitation, a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, Antibodies: A Laboratory Manual (1988).

Immunoassays can be used to determine presence or absence of an antibody or other biomarker in a sample as well as the quantity of an antibody or other biomarker in a sample. The amount of an antibody-target complex can be determined by comparing to a standard. A standard can be, for example, a known compound or another protein known to be present in a sample. It is understood that the test amount of antibody or other biomarker need not be measured in absolute units, as long as the unit of measurement can be compared to a control.

Many of the most useful antibody-based assays involve capturing the target antibody or antigen on a solid support in an antibody-antigen complex and then detecting the presence of the antibody-antigen complex. This can typically involve washing the solid support in washing buffer (e.g., PBS-Tween 20), blocking the solid support with an appropriate blocking buffer, washing the membrane in washing buffer, incubating the solid support with a secondary antibody (which recognizes the target antibody) conjugated to a detection element, such as an enzyme (e.g., horseradish peroxidase or alkaline phosphatase), radioactive molecule (e.g., ³²P or ¹²⁵I), or other signal-generating agent diluted in blocking buffer, washing the membrane in wash buffer, and detecting the presence of the antigen. Where the detection agent is an enzyme or other agent that needs a further step to generate a detectable signal, that further step would be performed before detecting the detectable signal. One of skill in the art would be knowledgeable as to the parameters that can be modified to increase the signal detected and to reduce the background noise.

In some forms, data generated by desorption and detection of antibodies and other biomarkers can be analyzed with the use of a programmable digital computer. The computer program analyzes the data to indicate the number of antibodies or other biomarkers detected, and optionally the strength of the signal and the determined molecular mass for each antibody or other biomarker detected. Data analysis can include steps of determining signal strength of an antibody or other biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set as zero in the scale.

A computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen, in another useful format, two or more spectra are compared, conveniently highlighting unique biomarkers and biomarkers that are up- or downregulated between samples. Using any of these formats, one can readily determine whether a particular biomarker is present in a sample.

ELISA, or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its enzymatic substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, α-glycerophosphate dehydrogenase, triose phosphate isomerase, glucose-6-phosphate dehydrogenase, glucoamylase and acetylcholinesterase. For descriptions of ELISA procedures, see Voller et al., J. Clin. Pathol. 31:507-520 (1978); Butler, Meth. Enzymol. 73:482-523 (1981); Maggio (ed.), Enzyme Immunoassay, CRC Press, Boca Raton, 1980; Butler, In: Structure of Antigens, Vol. 1 (Van Regenmortel, CRC Press, Boca Raton, 1992, pp. 209-259; Butler, In: van Oss, C. J. et al., (eds), Immunochemistry, Marcel Dekker, Inc., New York, 1994, pp. 759-803; Butler, J. E. (ed), Immunochemistry of Solid-Phase Immunoassay, CRC Press, Boca Raton, 1991); Crowther, “ELISA: Theory and Practice,” In: Methods in Molecule Biology, Vol. 42, Humana Press; New Jersey, 1995; U.S. Pat. No. 4,376,110, each of which is incorporated herein by reference in its entirety and specifically for teachings regarding ELISA methods.

Variations of ELISA techniques are known to those of skill in the art. In one variation, antibodies that can bind to proteins can be immobilized onto a solid support, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by, for example, the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label. In a related variation, antigen that can bind to an antibody of interest can be immobilized onto a solid support, such as a well in a polystyrene microtiter plate. Then, a test composition suspected of containing the antibody of interest can be added to the solid support. After binding and washing to remove non-specifically bound immunocomplexes, the bound antibody can be detected. Detection can be achieved by, for example, the addition of a second antibody specific for the antibody of interest, which is linked to a detectable element. The second antibody can be specific for the class of antibody to which the antibody of interest belongs, such as IgG or IgM.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunocomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means, rather than a direct procedure, can also be used. Thus, after binding of an antigen or antibody to the solid support, coating with a non-reactive material to reduce background, and washing to remove unbound material, the immobilizing surface is contacted with the control clinical or biological sample to be tested under conditions effective to allow immunocomplex (antigen/antibody) formation. Detection of the immunocomplex then requires a labeled secondary binding agent or a secondary binding agent in conjunction with a labeled third binding agent.

“Under conditions effective to allow immunocomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio. The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunocomplexes between the test sample and the originally bound material, and subsequent washing, the occurrence of even minute amounts of immunecomplexes can be determined.

To provide a detecting means, the second or third antibody can have an associated label to allow detection, as described elsewhere herein. This can be an enzyme that can generate color development upon incubating with an appropriate chromogenic enzymatic substrate. Thus, for example, one can contact and incubate the first or second immunocomplex with a labeled antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic enzymatic substrate such as urea and bromocresol purple or 2,2′-azido-di-(3-ethyl-benzthiazoline-6-sulfonic acid) (ABTS) and H₂O₂, in the case of peroxidase as the enzyme label. Quantitation can then be achieved by measuring the degree of color generation, e.g., using a visible spectra spectrophotometer.

Test strip assays can be used to detect antibodies or analytes such as antigens. In the antibody detection mode of test strip assay relevant for the disclosed methods, bait antigen is immobilized at one position on the test strip solid support and a control agent is immobilized at another position on the test strip solid support. In general, the test strip can be used to detect a target antibody by exposing the test strip to a sample and incubating to allow binding of target antibody to the immobilized antigen, washing the test strip to remove weakly bound or unbound antibodies, exposing the test strip to a detection agent that will bind to the target antibody, washing the test strip to remove weakly bound of unbound detection agent, and detecting the bound detection agent. In test strips for detecting antibodies, it is common and useful to use an anti-antibody antibody that can recognize the target antibody such as an anti-IgM or anti-IgG antibody for the species of the subject. The detection agent can include a detection element to facilitate detection of the detection agent. Detection of the detection agent can be by any suitable technique. For example, the detection element on the detection agent can be a directly detectable label (such as a fluorescent label or radioactive label, which directly produce or embody detectable signals) or an indirectly detectable label. An indirectly detectable label is a label that requires a further agent, element, and/or step to produce a detectable signal. For example, the detection element can be an enzyme (which will be used to produce a detectable signal via enzymatic reaction on an enzymatic substrate) or a tag to which a detectable label can bind. The control agent can be chosen to produce a detectable signal in the assay whether the target antibody is present or not. This control is used to show that the test strip and detection system are operable so that a negative detection of the target antibody is validated. A common and useful control agent an anti-antibody antibody that can bind a class of antibodies (such as IgM-class or IgM-class antibodies). In this way, the control agent will bind antibodies that are certain to be present in the sample (as well as, in most cases, the target antibody—although this is not required). Preferred modes for detection of the control are those that are used to detect the target antibody so that both the positive and control results are developed using the same procedures.

Lateral flow assays are also known as “dip-stick” or immunochromatographic strip tests. They are a popular platform for rapid tests and have been designed to detect viruses (e.g. influenza), as well as for home pregnancy tests. Lateral flow tests are used for the specific qualitative or semi-quantitative detection of many analytes including antigens from pathogens or antibodies against pathogens. Single or multi-analytes can be tested for simultaneously on the same strip. For human applications, any bodily fluid (e.g. urine, saliva, serum, plasma, or whole blood) can be used as a specimen. Test sensitivity and specificity can vary depending on the affinity and avidity of reagents produced. In the case of hepatitis B surface antigen, tests have claimed a sensitivity of 1.0 ng or less. The tests generally use colloidal gold, dye, or latex bead conjugates to generate a signal detectable by the user. An advantage of these types of tests in a diagnostic setting is that they are self-contained and do not require specific skills or training to perform or interpret. The assembled strips are prepared, dried and packaged and have a stable shelf-life when properly stored. To perform a test, the sample is placed in contact with the sample pad at one end of the strip. The detection agent (such as the anti-antibody antibody in the disclosed methods) binds to the antigen or antibody (anti-MBP84-104 antibody in the disclosed methods) in the sample and moves through the strip by capillary action. If specific analyte is present, a signal reagent binds to it, and a second antibody or antigen-immobilized as a line in the nitrocellulose-then captures the complex. If the test is positive, a colored line develops depending on the chromatogen employed in the test strip. Results are generally observed in 5 to 20 minutes. All tests include an internal positive control line that is used to validate the test result. The appearance of two lines, therefore, indicates a positive result, while a negative test produces only a single line.

In some forms of the disclosed methods, immunoblot assays can be used to detect antibodies to MBP84-104 in the disclosed samples. For immunoblot assays, in general, the sample would be placed in contact with a membrane (i.e., the solid support), such as nitrocellulose, PVDF or nylon, having immobilized MBP84-104, and antibodies to MBP84-104 would bind to the membrane. The membrane can then processed as described elsewhere herein. Basically, unbound or loosely bound antibodies are washed away and remaining available binding sites on the membrane are blocked with a blocking agent (e.g. casein, BSA, etc.). A detection agent, such as an anti-antibody antibody having a detection element, is then used to bind to the bound antibody, the membrane is washed and then a reporter agent is put in contact with the membrane. The reporter agent interacts or reacts with the detection element of the detection agent to produce a detectable signal. Alternatively, in some forms, the detection element can produce a detectable signal by itself and thus, a reporter agent is not necessary. Lastly, the detectable signal is detected by common procedures to those known in the art depending on what the detectable signal is.

Slot blot assays, also known as dot blots, are a form of immunoblot assay. Samples are administered directly to the membrane and the blocking, washing and detection steps would be the same as disclosed for immunoblots in general. In some forms, a plastic piece with holes sits directly above the membrane. There can be several pieces, each containing a different amount of holes and/or different size holes. Some pieces can comprise varying hole sizes. The sample can be added and suction from beneath the membrane directs the sample to contact the membrane only where a hole is present and thus, analytes only bind to the membrane in very specific locations. This allows the analytes to be concentrated in specific locations on the membrane which can allow for better detection. For example if specific analytes, which are in low concentrations in the sample, are spread out across an entire membrane the detection limit may prevent one from seeing a signal at the precise location of the analyte on the membrane. However, if these rare analytes are all bound in a specific location on the membrane, there would be enough analyte to be within the detection limit and thus a signal can be seen.

1. Arrays

The disclosed solid support can be configured as an array. In this regard, an array is a solid support with multiple different elements immobilized on the solid support in a predetermined pattern. Thus, a disclosed array includes, as an immobilized element, MBP84-104 immobilized at one or more predetermined locations on the solid support. The disclosed arrays generally will be an array of proteins and peptides. However, the disclosed arrays can also include immobilized nucleic acids. The solid support is something onto which a detection agent can be provided, (e.g., by attachment, deposition, coupling and other known methods). One or more detection agents may be immobilized on solid supports including, but not limited to glass (e.g., a chemically-modified glass slide), latex, plastic, membranes, microtiter, wells, mass spectrometer plates, beads (e.g., cross-linked polymer beads) or the like. An array can include, but is not limited to a plate, a chip, and/or a population of beads. A variety of array formats are known in the art and can be adapted to the inventive methods based on the descriptions provided in this application.

Solid supports for use in arrays can include any solid material to which an array element can be coupled, directly or indirectly. This includes materials such as acrylamide, cellulose, nitrocellulose, glass, polystyrene, polyvinylidene fluoride, filter paper (Whatman), Glass fiber filters (GFC) (A,B,C), polyethylimine coated GFCs, porous mylar or other transparent porous films, cellulose nitrate (CN) membrane, mixed cellulose ester membrane, cellulose acetate membrane, polyethersulfone (PES) membrane, PTFE membrane, ultrafiltration membranes of poly(vinyl chloride) (PVC), carboxylated poly(vinyl chloride) (CPVC), polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid supports can have any useful form including thin films or membranes, beads, bottles, dishes, fibers, woven fibers, shaped polymers, particles and microparticles. Preferred forms for a solid support are beads, membranes and a microtiter dish. The most preferred form of microtiter dish is the standard 96-well type.

The disclosed arrays can include between about 2, 3, 4, 5, 6, 7, 8, 9, 10, 100, 1,000, 2,000, 3,000, 4,000, 5,000, 6,000, 7,000, 8,000, 9,000, 10,000, 12,500 to 25,000, 50,000, 75,000, to about 100,000 distinct array elements, including values and ranges therebetween.

i. Protein Arrays

The disclosed solid support can be configured as a protein array. A protein array is a solid support with a plurality of different proteins or peptides immobilized on the solid support. The immobilized proteins or peptides are generally then used as bait for a binding partner the presence of which in a sample is to be assessed. Thus, a solid support configured as a protein array includes MBP84-104 as one of the proteins immobilized on the solid support. Standard techniques of microarray technology can be utilized to assess the presence antibodies specific for MBP84-104 in a sample. Protein microarray technology, which is also known by other names including: protein chip technology and solid-phase protein array technology, is well known to those of ordinary skill in the art and is based on, but not limited to, obtaining an array of identified peptides or proteins on a fixed solid support, binding target molecules or biological constituents to the peptides, and evaluating such binding. See, e.g., MacBeath and Schreiber, “Printing Proteins as Microarrays for High-Throughput Function Determination,” Science 289(5485):1760-1763, 2000.

ii. Capture Array

The disclosed solid support can be configured as a capture array. A capture array includes a plurality of capture tags immobilized to a solid support at identified or predetermined locations on the solid support. The immobilized capture tags are generally then used to capture a target molecule the presence of which in a sample is to be assessed. Thus, a solid support configured as a capture array includes MBP84-104 as one of the capture tags immobilized to the solid support. Each predetermined location on the solid support (referred to herein as an array element) has one type of capture tag (that is, all the capture tags at that location have the same structure). Each location will have multiple copies of the capture tag. The spatial separation of capture tags of different structure in the solid support allows separate detection and identification of target molecules that become associated with the capture tags. If a detection element is detected at a given location in a capture array, it indicates that the target molecule corresponding to that array element was present in the target sample.

Although preferred, it is not required that a given capture array be a single unit or structure. The set of capture tags may be distributed over any number of solid supports. For example, at one extreme, each capture tag may be immobilized in a separate reaction tube or container.

B. Treating and Administration

Some forms of the method involve treating a subject. Some forms of such treatment involve administering a compound or composition to a subject. In some forms, a subject can be treated by administering a ligand for voltage-gated Ca²⁺-channel α2δ1 (CACNA2D1 ligand), such as gabapentin or pregabalin.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care givers expertise, but that include the knowledge that the subject is ill, or will be ill, as the result of a condition that is treatable by the compounds of the invention. In preferred forms, a subject can be determined or assessed to be in need of treatment by detecting the presence of anti-MBP84-104 antibodies in the subject.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitiative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

In one aspect, the compounds described herein can be administered to a subject comprising a human or an animal including, but not limited to, a mouse, dog, cat, horse, bovine or ovine and the like, that is in need of alleviation or amelioration from a recognized medical condition.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need of treatment of neuropathic pain or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

Any of the compounds having the formula I can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E. W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

Reference herein to treating with a CACNA2D1 ligand refers, collectively and individually, to treating with a CACNA2D1 ligand, a composition comprising a CACNA2D1 ligand, a composition including a CACNA2D1 ligand, a composition consisting essentially of a CACNA2D1 ligand, an effective amount of a CACNA2D1 ligand, a composition comprising an effective amount of a CACNA2D1 ligand, a composition including an effective amount of a CACNA2D1 ligand, and a composition consisting essentially of an effective amount of a CACNA2D1 ligand. In some forms, in the context of administration to or treatment of a subject, to a composition consisting essentially of a component or components can refer to a composition that does not contain or include more than a de minimis amount (e.g., an ineffective amount) of a therapeutic agent of any type or purpose, a therapeutic agent of the same type or purpose, or a therapeutic agent for treating the same disease or condition as the component or components. Thus, for example, in some forms, in the context of administration to or treatment of a subject, to a composition consisting essentially of a CACNA2D1 ligand can refer to a composition that does not contain or include more than a de minimis amount (e.g., an ineffective amount) of a therapeutic agent of any type or purpose, a therapeutic agent of the same type or purpose, or a therapeutic agent for treating the same disease or condition as the CACNA2D1 ligand.

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

### EXAMPLES

Materials and Methods

1. General reagents, antibodies and cells.

All reagents were purchased from Sigma-Aldrich and ThermoFisher Scientific unless indicated otherwise. A rabbit polyclonal MBP antibody (AB980), a broad-spectrum hydroxamate MMP inhibitor (GM6001), and both the HRP-conjugated goat antihuman IgG (#401455) and IgM antibodies (#401905) were from EMD Millipore. HRP-conjugated goat anti-rat IgM (#3020-05) was from Southern Biotech. The HRP-conjugated goat anti-rat IgG (#112-035-175) and the fluorescent Mca-PLGL-Dpa-AR-NH₂ MMP substrate were from Jackson ImmunoResearch and R&D Systems, respectively. A 3,3′,5,5′-tetramethylbenzidine substrate (TMB/E) and IgG & protease-free BSA (a 30% solution) were from Surmodics and US Biological, respectively.

2. MBP and peptides.

Human intact MBP (a 18.5 kDa isoform) was from Meridian Life Science. The synthetic wild-type (MBP84-104-WT; ENPVVHFFKNIVTPRTPPPSQ; SEQ ID NO:11) and scrambled (MBP84-104-SCR; EFPHIKVTVVTPRNGFPNSPP; SEQ ID NO:12) peptides (97-99% purity) were synthesized by GenScript and protected from exoprotease degradation by N- and C-terminal biotinylation and amidation, respectively. Peptides are numbered according to the human MBP sequence (GenBank # AAH08749).

3. CCI mononeuropathy model, pain testing and sample collection.

Fifty-six adult female and sixteen male Sprague-Dawley rats (200-225 g) were obtained from Envigo Labs and housed in a temperature-controlled room (22° C.), on a 12-h light/dark cycle with free access to food and water. Animals were anesthetized with 4% isoflurane in oxygen (Aerrane; Baxter) and then the common sciatic nerve was exposed unilaterally at the mid-thigh level. The nerve received three loosely constrictive chromic gut ligatures to produce CCI (Liu et al. J Neuroinflammation 9:119, (2012); Shubayev and Myers, Brain Res 855:83-89, (2000); Hong et al. Brain Behav Immun 60:282-292, (2017)). Behavioral testing was conducted at fixed times between 8:00 a.m. and 2:00 p.m. The thresholds for mechanical allodynia were measured using von Frey filaments (Stoelting), ranging from 0.41 to 15.2 g. Rats were positioned in a Plexiglas enclosure on top of a wire mesh surface and habituated to the environment prior to testing. Each filament was applied perpendicularly on the mid-hind paw according to the Dixon up-down method. A 50% probability of withdrawal threshold was calculated as previously described (Chaplan et al. J Neurosci Methods 53:55-63, (1994)) and expressed in grams. Withdrawal thresholds for both hind paws were determined and averaged. At days 1-28 post-CCI and in naïve or sham-operated animals, sciatic nerve, blood and urine samples were collected from the same cohort of rats. Sciatic nerve samples were snap-frozen in liquid N2 and stored at −80° C. until use. Blood aliquots (1-2 ml, each) were obtained by cardiac puncture and collected in tubes without anti-coagulant. Blood samples were allowed to clot for 30 min at ambient temperature, centrifuged (2,000×g; 10 min; 4° C.) and the supernatant serum was stored at −80° C. Urine sample aliquots (0.2-0.4 ml) were collected in awake animals, just prior to behavioral testing: to assess the MMP activity, the samples were readily placed on ice for a few min and then cleared by centrifugation (2,000×g; 10 min; 4° C.). Cleared aliquots were equilibrated in 50 mM HEPES, pH 7.5, containing 10 mM CaCl₂, 0.5 mM MgCl₂ and 10 μM ZnCl₂, using a desalting spin-column and immediately used in the MMP activity tests. In a separate group of animals, sciatic nerves were collected in RNA-later and stored at −20° C. for the qRT-PCR analyses. Animals were sacrificed using Beuthanasia (150 mg/ml; i.p., Schering-Plough Animal Health). All animal procedures were performed according to the PHS Policy on Humane Care and Use of Laboratory Animals with the experimental protocol approved by the Institutional Animal Care and Use Committee at the VA San Diego Healthcare System, and complied with ethical guidelines of the International Association for the Study of Pain.

4. Taqman qRT-PCR.

Taqman primers and a probe containing 5′-FAM reporter for rat TIMP-1 (GenBank, NM_053819) were from Applied Biosystems (cat. # Rn01430873g1). Primers and probes for MMP-9 (GenBank, NM_031055) and glyceraldehyde 3-phosphate dehydrogenase (GAPDH; GenBank, X02231) were from Biosearch Technologies (Liu et al. J Neuroinflammation 9:119, (2012); Shubayev et al. Mol Cell Neurosci 31:407-415, (2006)). Total RNA was extracted using TRIzol and purified on an RNeasy mini column (Qiagen). The RNA purity was estimated by measuring the A_{260/280} and the A_{260/230} ratios. The samples were treated with RNasefree DNAse I (Qiagen). cDNA was synthesized using a first strand cDNA kit (Roche). Gene expression levels were measured in a Mx3005P (Agilent) using 50 ng cDNA and 2×Taqman Universal PCR Master Mix (Applied Biosystems) with a one-step program: 95° C., 10 min; 95° C., 30 sec; 60° C., 1 min for 50 cycles. Using the injured sciatic nerve cDNA samples, primers (Biosearch Technologies) and Taqman probes for MMP-9 (Roche) and TIMP-1 (Applied Biosystems) were earlier optimized to reach the amplification efficiency of 100.1-100.3% (Shubayev et al. Mol Cell Neurosci 31:407-415, (2006)). GAPDH was used as a normalizer; its expression changes were insignificant in the injured relative to naive nerves. Samples without cDNA (a no template control) showed no contamination. Relative mRNA levels were quantified using the comparative delta Ct method (Livak and Schmittgen Methods 25:402-408, (2001)). The foldchange between experimental and control samples was determined using the Mx3005P software.

5. Protease activity assay.

The cleavage assay was performed in a total volume of 0.2 ml in triplicate in wells of a 96-well plate using the fluorescent Mca-PLGL-Dpa-AR-NH₂ peptide substrate (1 μM) and the 50 μl urine aliquots equilibrated in MMP buffer, pH 7.5 (50 mM HEPES buffer, pH 7.5, containing 10 mM CaCl₂), 0.5 mM MgCl₂ and 10 μM ZnCl₂). Where indicated, GM6001 (10 μM) was co-incubated for 30 min at ambient temperature with the urine samples to inactivate MMPs. Initial reaction velocity was monitored continuously at λₑₓ=320 nm and λₑₘ=400 nm using a fluorescence spectrophotometer. Data are means±SEM from several independent experiments. Protein concentrations in the dialyzed urine samples were determined using the Bradford assay and made even by sample dilution in MMP buffer, pH 7.5.

6. MMP-9 purification using gelatin-Sepharose beads.

The proteins were extracted for 1 h at 4° C. from the sciatic nerve samples using 50 mM Tris-HCl buffer, pH 7.4, containing 150 mM NaCl, 1% Triton X-100, 0.1% SDS, 10 mM EDTA, the protease cocktail inhibitor and 1 mM phenylmethylsulfonyl fluoride. The protein concentration of the extracts was determined using a Coomassie Protein Assay, and then adjusted to reach 1 mg/ml. The extracts aliquots (100 μg total protein, each) were 10-fold diluted using the above buffer lacking Triton X-100 and SDS, and then allowed to bind to gelatin-Sepharose beads for 16-18 h at 4° C. Following extensive washing, the bound material was eluted using 2×SDS sample buffer (50 μl).

7. Gelatin zymography.

The dialyzed rat urine samples equilibrated in MMP buffer, pH 7.5, the crude nerve extracts and the MMP-9 samples isolated from sciatic nerve were analyzed by gelatin zymography in a 10% acrylamide-0.1% gelatin gel. Gels were next processed as described previously to visualize the clear gelatinolytic activity bands (Liu et al. J Neuroinflammation 9:119, (2012)). Where indicated, gels were incubated in 20 mM EDTA to inactivate MMP activity.

8. Human participants, pain evaluation and sample collection.

Fifteen female patients were recruited from two different studies, both approved by the Institutional Review Board at UCSD, where the studies were conducted. Five patients with multiple sclerosis (MS) were recruited from the Multiple Sclerosis clinic, the Department of Neurology, UCSD. Each subject received a detailed history and physical examination, including average pain intensity for the past month quantified by numeric rating scale, followed by quantitative sensory tests performed in the area of pain in subjects with significant chronic pain; if no pain was present at the moment, the tests were performed on the dominant lower extremity. Quantitative sensory tests included dynamic mechanical allodynia [pain intensity to tangential application of a foam pain brush, quantified by visual analog scale (VAS), 0-100 mm], static mechanical allodynia (pain intensity to a 3 sec application of a 5.18 g von Frey hair quantified by VAS), thermal hyperalgesia [pain intensity to a 1 sec application of a 36×42 mm thermal probe (TSA-II NeuroSensory Analyzer, Medoc Advanced Medical Systems) heated to 45° C. applied to the skin quantified by VAS], thermal pain threshold (temperature at which subject reported pain when the thermal probe was heated gradually from 32° C. to 50° C. at a rate of 1.5° C./sec), and pressure pain threshold (force, in lb, at which subjects reported pain when the 1 cm rubber tip of a Wagner FPK manual pressure algometer was applied to the skin). As a control, we recruited two healthy female volunteers who did not present any signs of MS or any other acute disease conditions. For comparison, eight patients with fibromyalgia syndrome (FMS) were enrolled from the primary care and chronic pain clinics at UCSD. A diagnosis of FMS was confirmed by study staff. None of the subjects showed any clinical sign of active infection at the time of sample collection. Demographic and clinical data from subjects with MS and FMS are summarized in Table 1. Serum samples were collected from all subjects and handled using standard procedures and then stored at −80° C.

9. ELISA of the anti-MBP84-104 epitope IgG and IgM antibodies in serum samples.

The wells of a 96-well Maxisorp ELISA plate were coated for 18 h at 4° C. with ExtrAvidin (3 μg/ml in 0.125 ml 15 mM bicarbonate buffer, pH 9.6). Non-specific binding was blocked for 1 h at 37° C. using 1% IgG & protease-free BSA (0.4 ml) in 50 mM Tris-HCl buffer, pH 7.8, containing 1 M NaCl and 0.1% Tween-20 (TBS/T). After six washes (5 min, each; 500-700 rpm) in TBS/T at ambient temperature, the biotin-labeled MBP84-104-WT and -SCR peptides (5 μg/ml in 0.1 ml TBS/T-1% BSA, each) were added to the wells and incubation was continued at 4° C. for an additional 16-18 h. The follow-on procedures were carried out at ambient temperature and gentle agitation (500-700 rpm). After six washes (5 min, each) in TBS/T, rat or human serum samples (diluted 1:50 in 0.1 ml TBS/T-1% BSA) were allowed to bind to the MBP peptide-coated wells for 3 h. Following extensive washes in TBS/T (5 min, each), the secondary HRP-conjugated species-specific IgG or IgM antibodies (diluted 1:10,000 in 0.1 ml TBS/T-1% BSA, each) were added to the wells for 1 h. After extensive washes in TBS/T (5 min, each), and then with H₂O, the TMB/E substrate (0.1 ml) was added to the wells. The reaction was stopped by adding 1 M H₂SO₄ (0.1 ml) and the resulting A₄₅₀ value was measured using a plate reader. Data are means±SE from at least 3 individual experiments performed in triplicate.

For ELISA using intact MBP as bait, full-length MBP (3 μg/ml in 0.125 ml 15 mM bicarbonate buffer, pH 9.6) was added to the wells. Wells coated with BSA (3 μg/ml) served a control. TBS/T-1% BSA (0.4 ml) was used to block the non-specific binding. The plates were next incubated for 3 h at ambient temperature with the rat or human serum samples (diluted 1:50 in 0.1 ml TBS/T-1% BSA). The follow-on steps were as described above.

10. Data analyses.

Statistical analyses were performed using SPSS 16.0 software by a two-tailed, unpaired Student's t-test for comparing two groups, or analyses of variance (ANOVA) for repeated measures for comparing three or more groups, followed by the Bonferroni post-hoc test, unless specified otherwise. P<0.05 values were considered significant.

### Example 1. MMP-9 Activity in the Injured Sciatic Nerve in Rats

In the early phase of painful PNS injury in vivo, MMP-9 is believed to initiate the proteolytic fragmentation of MBP and to release the MBP fragments, including the immunodominant, algesic MBP84-104 epitope (Liu et al. J Neuroinflammation 9:119, (2012); Shiryaev et al. PLoS One 4:e4952, (2009); Hong et al. Brain Behav Immun 60:282-292, (2017)). The continuing MMP-9 activity in the injured nerve microenvironment would increase the MBP fragment release, thus providing a means for the raise of the anti-algesic fragment autoantibodies. However, the level of MMP-9 in the late, chronic phase of painful nerve injury has not been previously assessed.

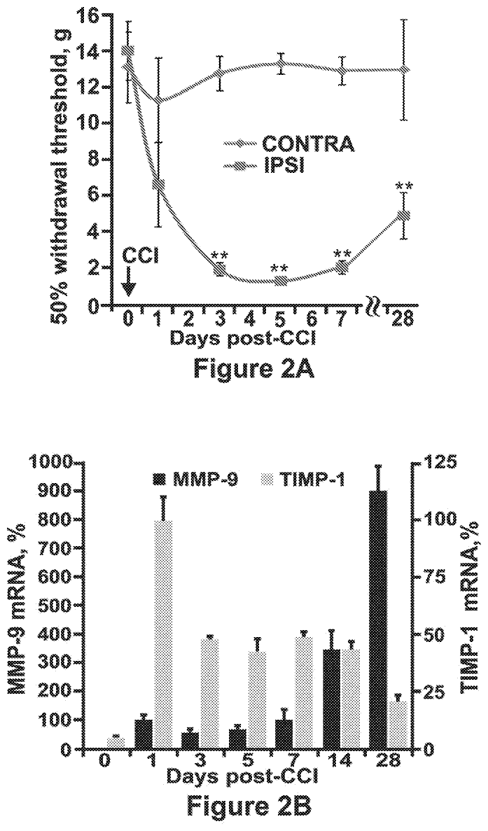

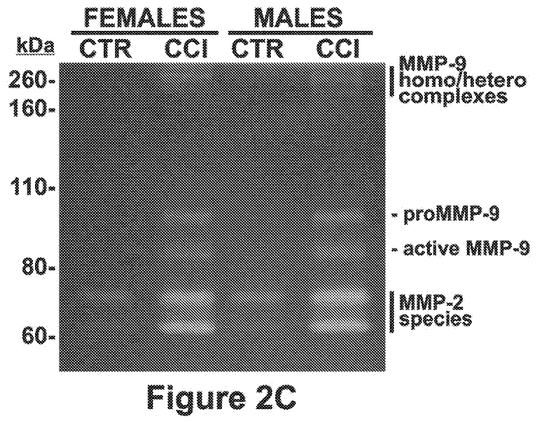

For this purpose, sciatic nerve of female rats was exposed unilaterally to receive three loosely constrictive ligatures to produce CCI mononeuropathy (Liu et al. J Neuroinflammation 9:119, (2012); Hong et al. Brain Behav Immun 60:282-292, (2017)). Using von Frey behavioral testing, it was determined that the significant mechanical hypersensitivity (allodynia) at day 1 was sustained, at least, to day 28 post-CCI, as there was a prominent reduction in the mechanical stimulus required to evoke a withdrawal response in the hind paw ipsilateral to injury (˜5 grams), as compared to the hind paw contralateral to injury (12-13 grams) (FIG. 2A).

There is a consensus that the MMP-9-TIMP-1 ratio largely determines the net proteolytic activity of the MMP-9 enzyme. Taqman qRT-PCR was used to quantify the MMP-9-TIMP-1 expression ratio in the nerve between day 0 (prior to injury) and day 28 post-CCI in female rats (FIG. 2B). In the rat naïve nerve (day 0), the MMP-9 mRNA level was exceedingly low. The MMP-9 mRNA level at the nerve injury site increased in the bi-phasic manner peaking at day 1, and then again at day 28 post-CCI. In contrast, the TIMP-1 mRNA level was high in rat naïve nerve, consistent with our previous reports (Kim et al. PLoS One 7:e33664, (2012); Liu et al. J Neuroinflammation 9:119, (2012); Chernov et al. J Biol Chem 290:11771-11784, (2015); Chattopadhyay and Shubayev, Glia 57:1316-1325, (2009); Kobayashi et al. Mol Cell Neurosci 39:619-627, (2008); Shubayev et al. Mol Cell Neurosci 31:407-415, (2006)). Relative to naïve nerve, TIMP-1 mRNA peaked at day 1 post-CCI but then declined over time. At day 28 post-CCI, the levels of TIMP-1 and MMP-9 were reduced ˜5-fold and increased ˜9-fold relative to day 1, respectively. These findings suggested that in the injured nerve microenvironment the MMP-9-TIMP-1 ratio shifted in a favor of MMP-9 activity and that there was a ˜45-fold increase in the protease-inhibitor ratio at day 28 post-CCI relative to day 1.

To corroborate these findings and to test if MMP-9 upregulation induced by CCI in females are recapitulated in males, we analyzed MMP-9 activity in sciatic nerve collected from male and female rats at day 28 post-CCI. Nerve extracts were first incubated with gelatin-Sepharose beads to purify MMP-9 prior to analysis by gelatin zymography. MMP-9 was not detected in the naïve nerve (CTR, contralateral to injury) in female or male rats (FIG. 2C). In agreement with our earlier reports (Kim et al. PLoS One 7:e33664, (2012); Chattopadhyay and Shubayev, Glia 57:1316-1325, (2009); Shubayev et al. Mol Cell Neurosci 31:407-415, (2006); Chattopadhyay et al. Brain Behav Immun 21:561-568, (2007)), MMP-9 in the injured nerve (CCI, ipsilateral to injury) was dramatically up-regulated and activated similarly in both female and male rats. Indeed, no significant difference was observed in both the intensity and the species of MMP-9 bands between the female and male animals. The gelatinolytic activity bands in the injured nerve samples corresponded to the known species of MMP-9, including the 92 kDa proenzyme, the 84 kDa active enzyme and multiple 200-260 kDa MMP-9 homo/heterodimers (Kim et al. PLoS One 7:e33664, (2012); Nagase and Murphy, Cardiovasc Res 69:562-573, (2006); Piccard et al. J Leukoc Biol 81:870-892, (2007)).

In sum, RT-PCR and gelatin zymography data suggest an increase in the MMP-9 activity in the late, chronic phase of painful nerve injury such as day 28 post-CCI. This increase may contribute to the continuing release of the algesic MBP fragment(s) and, consequently, to the sustained pain state in both females and males, and, in addition, to stimulate and then to support the persistent raise of the MBP autoantibodies in the injured animals.

### Example 2. Post-Injury Excretion of MMP Excess in the Rat Urine

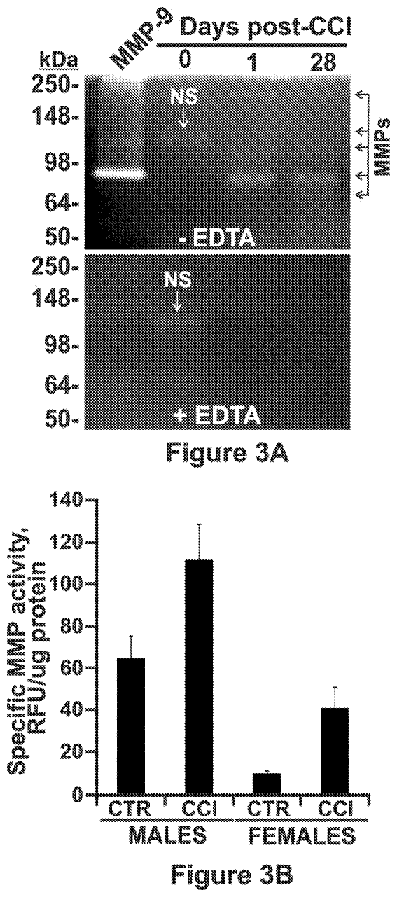

Glycoproteins, including MMP-9 and some other MMPs, are normally excreted via the urine (Moses et al. Cancer Res 58:1395-1399, (1998); Marimuthu et al. J Proteome Res 10:2734-2743, (2011)). MMP-9 is a glycoprotein with multiple O-glycosylation sites in the linker region between the catalytic and hemopexin domains, and two N-glycosylation sites in the prodomain (Duellman et al., Traffic 16:1108-1126, (2015); Van den Steen et al. J Biol Chem 281:18626-18637, (2006); Vandooren et al., Crit Rev Biochem Mol Biol 48:222-272 (2013)). To test if an increase in MMPs in the injured nerve was followed by an elevated MMP excretion in the urine and if it was distinct in female and male rats, we examined the status of the gelatinolytic MMPs in the rat urine using gelatin zymography. There was a significant increase in the urinary MMPs at day 1 post-CCI relative to day 0 in female rats (FIG. 3A). Similar results were obtained with the male urine samples (data not shown). The elevated MMP activity sustained in the urine until day 28 post-CCI. To confirm the MMP identity of the gelatinolytic bands, we used EDTA, a metal chelator and a general inhibitor of zinc-MMPs. EDTA repressed all of the MMP-related gelatinolytic bands in the samples, except a single EDTA-resistant band in the day 0 samples suggesting the presence of a minor non-MMP gelatinolytic activity in the normal rat urine.

To support these findings, we assessed the MMP activity in the rat urine samples using the Mca-PLGL-Dpa-AR-NH₂ fluorescent peptide as a broad-specificity MMP cleavage substrate. Because impurities in the crude samples interfered with the fluorogenic substrate assay (data not shown), we dialyzed the urine samples against the MMP buffer, pH 7.5 prior to the assay. The dialyzed samples were incubated with the Mca-PLGL-Dpa-AR-NH₂ in the presence and absence of GM6001, a broad-range hydroxamate inhibitor of MMPs. Activity measurements demonstrated that, relative to the naïve animals, the MMP activity in a similar fashion in both CCI male and female rats (50 RFU/—g proteins), although the naïve control male group exhibited a higher MMP activity background (FIG. 3B).

Overall, an enhanced MMP excretion in the urine corroborates the up-regulation of MMPs in the injured nerve and supports both the continuing MMP proteolysis of MBP and the favorable conditions for the induction of the autoantibodies in the traumatized animals.

### Example 3. Anti-Algesic MBP Epitope IgM Autoantibodies as a Hallmark of Chronic Neuropathic Pain in Female Rats

In female rats, a single intra-neural injection of the synthetic algesic MBP84-104 peptide into an intact sciatic nerve causes a robust and long-lasting mechanical allodynia in female rats (Liu et al. J Neuroinflammation 9:119, (2012); Ko et al. Brain Behav Immun 56:378-389, (2016); Hong et al. Brain Behav Immun 60:282-292, (2017)). Allodynia, however, was diminished in immunodeficient rats, indicating an important role of adaptive immunity in the MBP-induced painful nociception (Liu et al. J Neuroinflammation 9:119, (2012)). Based both on these results and the persistent presence of the elevated MMP activity post-injury, we hypothesized that CCI mononeuropathy causes the continued proteolytic release of the algesic, immunodominant MBP84-104 epitope from the damaged myelin sheath and that this persistent epitope release facilitates autoimmunity leading to the circulating anti-MBP84-104 autoantibodies.

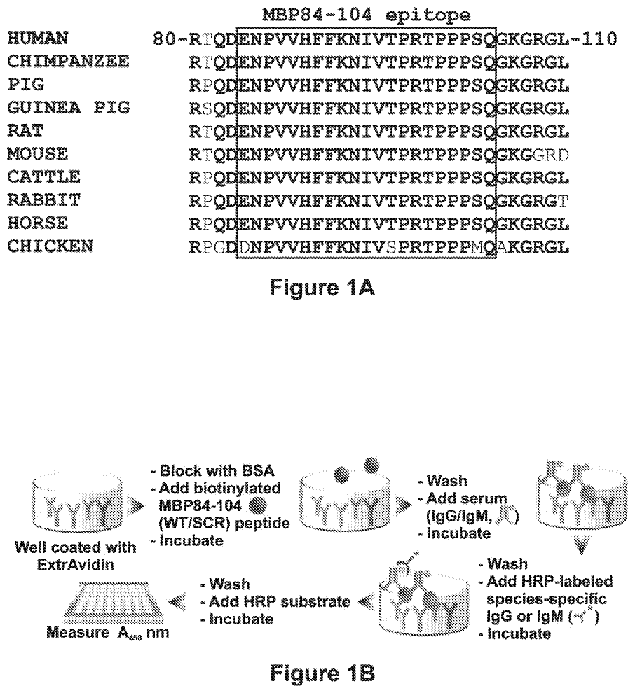

To test this hypothesis, we developed a peptide-based ELISA methodology that employed the biotin-labeled synthetic MBP84-104 peptide as bait and the horseradish peroxidase (HRP)-labeled goat anti-rat IgG and IgM antibodies (FIG. 1B). The scrambled (SCR) peptide was used as a control. This ELISA approach allowed us to assess, both quantitatively and in a highly reproducible fashion, the levels of the specific anti-MBP84-104 epitope IgM and IgG antibodies in the rat serum samples.

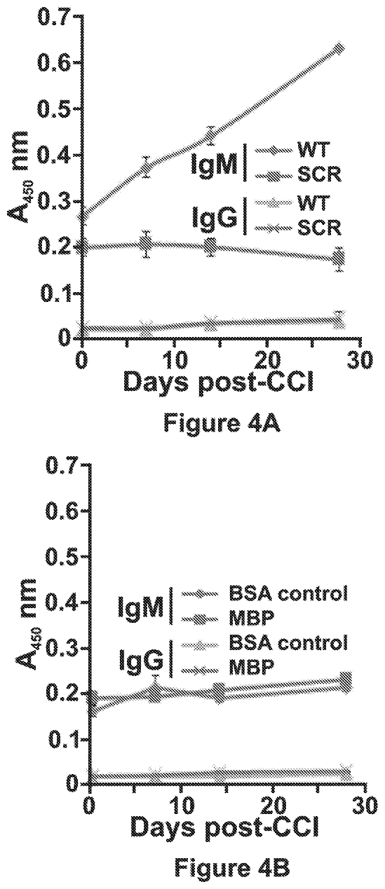

In female rat serum, the ELISA measurements revealed that the level of the circulating anti-MBP84-104 IgM autoantibody pool, but not the IgG autoantibodies, significantly increased, in a time-dependent manner, in the rat serum after nerve injury. At day 28 post-CCI, the level of the specific IgM antibodies increased ˜7-fold relative to the control serum (FIG. 4A). Because the immunodominant MBP84-104 epitope region is hidden in the full-length intact MBP fold, a similar increase was not observed with the full-length MBP bait—the A₄₅₀ values were low and there was no significant difference between full-length MBP and a BSA control with neither IgM nor IgG antibodies (FIG. 4B). These results indicate that the ELISA methodology we designed can be employed as a reliable tool to identify seropositivity for the anti-algesic MBP84-104 epitope autoantibodies in the rat serum.

### Example 4. Autoantibodies Against the Algesic MBP Epitope are not Induced in Male Rats Post-CCI

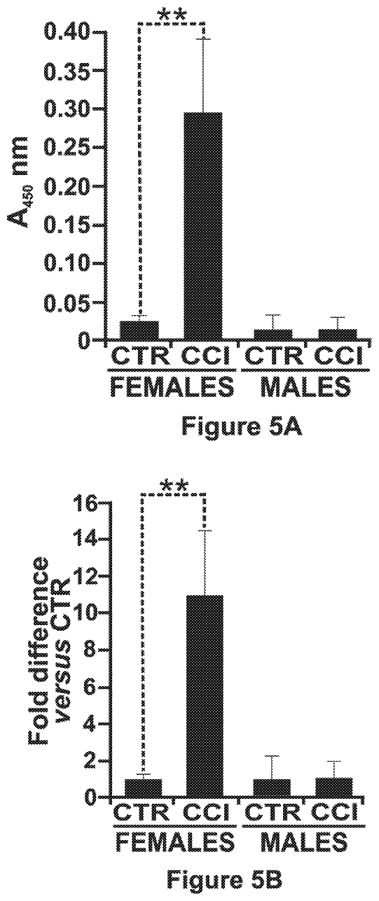

Female rats alone have been routinely used in our earlier studies (Kim et al. PLoS One 7:e33664, (2012); Liu et al. J Neuroinflammation 9:119, (2012); Ko et al. Brain Behav Immun 56:378-389, (2016); Hong et al. Brain Behav Immun 60:282-292, (2017); Shubayev et al. Neural Regen Res 11:890-891, (2016); Chernov et al. J Biol Chem 290:1171-11784, (2015); Nishihara et al. J Biol Chem 290:3693-3707, (2015)). In contrast, male animals were primarily employed in the numerous studies by many others. Here, we tested if the pain mechanisms induced by CCI in females are reiterated in males. Von Frey tests demonstrated that CCI induced mechanical allodynia in male rats and that the response curve over time was similar to that in female rats, albeit consistent with the data by others (Nicotra et al. Front Behav Neurosci 8:40, (2014)), pain was less pronounced in males (data not shown). Earlier we demonstrated that the upregulation of active MMP-9 in the injured nerve (FIG. 2C) and the excretion of the MMP activity excess in the urine (FIG. 3B) were comparable in male and female rats with CCI. However, in a sharp contrast to females, the level of the circulating anti-MBP84-104 epitope IgM antibodies was significantly less noticeable in male rats as compared with females (FIGS. 5A and 5B). The serum levels of the anti-MBP84-104 IgM antibodies at day 28 post-CCI in female rats was elevated ˜10-fold relative to the naïve control, whereas no significant changes were observed at this time point between the naïve and post-CCI male rats. These observations support the earlier finding by us and others that there is sexual dimorphism in the painful nociception mechanisms and that adaptive immunity, and B and T cells play a more definitive role in neuropathic pain in females versus males (Shubayev et al. Neural Regen Res 11:890-891, (2016); Sorge et al. Nat Neurosci 18:1081-1083, (2015); Nicotra et al. Front Behav Neurosci 8:40, (2014)).

### Example 5. Autoantibodies Against the Algesic MBP Epitope are a Marker of Demyelinating Neuropathy in Humans

To test if the peptide-based ELISA methodology we developed is applicable to clinic, we evaluated a limited number of serum samples obtained from fifteen human female subjects (Table 1): five female patients with MS, a chronic autoimmune demyelinating disease, often accompanied by neuropathic pain and autoantibodies against the MBP84-104 peptide (Solaro and Messmer, Drugs 70:1245-1254, (2010); Wucherpfennig et al. J Clin Invest 100:1114-1122, (1997)); eight female patients with FMS, a prevalent medical condition characterized by chronic widespread pain, diffuse tenderness and heightened pain response to pressure, not known to produce autoantibodies against MBP84-104 peptide (Sluka and Clauw, Neuroscience 338:114-129, (2016); Wolfe et al. Arthritis Care Res (Hoboken) 62:600-610, (2010)); and two healthy female volunteers.

TABLE 1
Demographics and Pain Evaluation Data of Patients
with MS and FMS, and Healthy Volunteers.

| Demographics and pain evaluation | MS (n = 5) | | FMS (n = 8) | | Healthy (n = 2) | |
|---|---|---|---|---|---|---|
| Gender | Female | | Female | | Female | |
| Age in years (range) | 40.6 | (29-63) | 44 | (24-64) | 53 | (50-56) |
| Disease duration, in years (range) | 12.6 | (3-27) | ND | | NA | |
| Pain sensitivity evaluation | | | | | | |
| (average, range) | | | | | | |
| NRS, pain severity | 7.7 | (5-10) | 4 | (2-9) | 0 | |
| VAS, dynamic mechanical allodynia | 0.2 | (0-1) | ND | | 4 | (0-14) |
| VAS, static mechanical allodynia | 3.8 | (0-11) | 6 | (5-7) | 011.2 | (0-23) |
| VAS, thermal hyperalgesia | 4 | (0-13) | 6 | (1-10) | 0 | |
| VAS, pressure pain threshold (lb) | 9.6 | (6-16) | 15 | (6-25) | 9.9 | (7.2-13) |

| | | | | | |
|---|---|---|---|---|---|
| Heat pain threshold (° C.) | 47.8 | (44.8-50) | ND | 48.5 | (45.9-50) |

ND, not determined; NA, not applicable; NRS, numeric rating scale; VAS, visual analog scale.

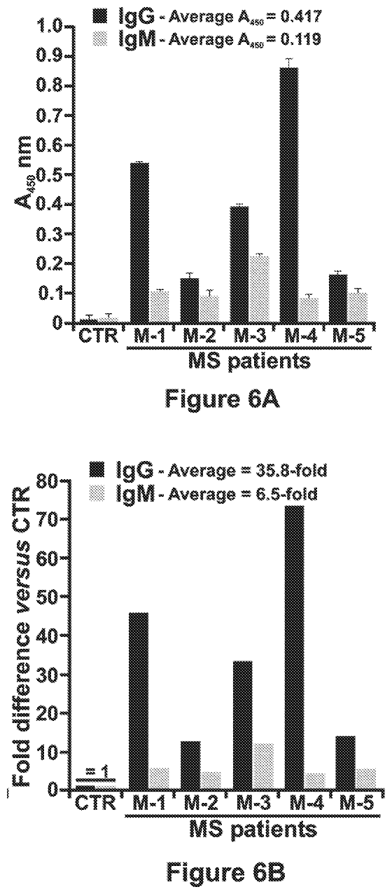

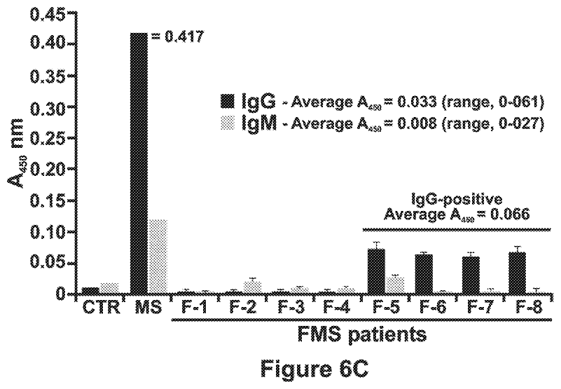

In all MS patients, the serum level of the specific anti-algesic MBP84-104 peptide IgG antibodies was elevated 12.6-73.3-fold (average=35.8-fold), relative to the healthy volunteers (FIGS. 6A and 6B). Similarly, a significant, albeit less pronounced, 4.4-12.1-fold (average=6.5-fold), increase in the IgM antibodies to MBP84-104 was recorded in MS patients (FIGS. 6A and 6B).

In contrast to MS, the serum of FMS patients demonstrated a dramatically lower level of the MBP84-104 IgG autoantibodies. On average, the IgG levels were ˜13-fold lower in patients with FMS (average A₄₅₀=0.033) compared with those in MS samples (average A₄₅₀=0.417; FIG. 6C). Four of eight (50%) FMS patients exhibited a measurable, albeit very low, level of seropositivity, corresponding to a ˜5.5-fold increase (average A₄₅₀=0.066) relative to the healthy volunteers (average A₄₅₀=0.012). The other 4 patients with FMS were seronegative. The level of the specific IgM autoantibodies against the MBP84-104 peptide was exceedingly low or even below detection level in all FMS samples (FIG. 6C).

Notably, the peptide-based ELISA methodology we developed delivers reproducible measurements of the specific circulating IgG and IgM autoantibodies against the algesic MBP epitope in both rat and human serum of subjects experiencing pathological pain and a focalized myelin damage. These anti-MBP84-104 autoantibodies are prevalent in MS, a demyelinating disease, and they are not common in all type of chronic pain conditions or in healthy subjects.

In sum, our results highlighted the presence of sexual dimorphism in painful neuropathy related to autoimmunity, and implicate female-specific seropositivity for the anti-algesic MBP peptide autoantibodies as a valuable novel biomarker of demyelinating painful pathologies.

### Discussion

The release of myelin autoantigens may occur in the absence of demyelinating diseases. Our research strongly suggests a major contribution of the released cryptic epitopes of myelin auto-antigens to states of chronic pain in the absence of demyelinating disease. Because it was not known if the proteolytic release of the cryptic pro-algesic MBP84-104 epitope after traumatic nerve damages is followed by the raise of the anti-MBP epitope autoantibodies and if these autoantibodies do or do not directly contribute to focal painful mononeuropathy, we developed an peptide-based ELISA methodology to measure the level of the circulating autoantibodies against the algesic MBP84-104 peptide in human and rat sample (FIG. 1B).

Mechanical allodynia is a common manifestation of PNS lesions leading to the maladaptive activity of mechanosensory afferents and the myelin sheath (Devor, Brain Res 196:115-128, (2009); Djouhri and Lawson, Brain Res Brain Res Rev 46:131-145, (2004); Campbell et al. Pain 32:89-94, (1988); Woolf and Doubell, Curr Opin Neurobiol 4:525-534, (1994)). The algesic MBP epitopes are involved in the painful autoimmune demyelinating pathologies, including Guillain-Barre syndrome and MS, and experimental mononeuropathies, including CCI and nerve crush (Kim et al. PLoS One 7:e33664, (2012); Liu et al. J Neuroinflammation 9:119, (2012); Hong et al. Brain Behav Immun 60:282-292, (2017)). Because of the focal myelin membrane destabilization (e.g., in diabetes and chemotherapy, by viral pathogens and nerve entrapment or compression), proteolysis of myelin sheath leading to the release of the algesic MBP epitopes may also occur without extensive primary demyelination. To this end, we also recorded the algesic MBP peptide release prior to demyelination (Liu et al. J Neuroinflammation 9:119, (2012)). Intriguingly, the intrasciatic injection of the algesic MBP84-104 peptide in females produced robust allodynia in the absence of overt neuropathology or widespread neuroinflammation (Liu et al. J Neuroinflammation 9:119, (2012); Ko et al. Brain Behav Immun 56:378-389, (2016)). Innate immunity response and the raise of autoantibodies may follow the release of the algesic immunodominant MBP peptidic epitopes (Polman and Killestein, J Neurol Neurosurg Psychiatry 77:712, (2006). Thus, the presence of autoantibodies against neural antigens is frequently attributed to pathological pain states (McMahon et al. Nat Rev Neurosci 16:389-402, (2015); Bennett and Vincent, Neurology 79:1080-1081, (2012); Wigerblad et al. Ann Rheum Dis 75:730-738, (2016); Sorkin et al. Brain Res 930:67-74, (2002); Sorkin, Pain Med 1:296-302, (2000); Mifflin and Kerr, J Neurosci Res 95:1282-1294, (2017); Klein et al. Neurology 79:1136-1144, (2012); Xiao et al. Pain 69:145-51, (1997); Goebel, Autoimmun Rev 15:552-557, (2016)), but the quantitative assessment of the anti-MBP autoantibodies in painful nociception has not been accomplished. To this end, this study provides the first evidence for the presence of the circulating autoantibodies against the algesic portion of MBP in the experimental focal peripheral mononeuropathy such as CCI-related mechanical allodynia in female rodents.

Evidence suggests that in the injured nerve the enhanced MMP activity (from which the pro-inflammatory MMP-9 activity is most well recognized) degrades MBP and releases its cryptic epitopes such as the algesic immunodominant 84-104 region (Kim et al. PLoS One 7:e33664, (2012); Liu et al. J Neuroinflammation 9:119, (2012); Shubayev and Myers, Brain Res 855:83-89, (2000); Kobayashi et al. Mol Cell Neurosci 39:619-627, (2008); Shubayev et al. Mol Cell Neurosci 31:407-415, (2006)). Prior to injury, MMP-9 levels are miniscule in the naïve sciatic nerve. In contrast, after the injury MMP-9 is upregulated in a matter of hours and then the MMP-9 enzymes is continually present in the injured nerve microenvironment and its level increases even further at the late, day 28, chronic phase of allodynia. Our data demonstrated that at this late phase MMP-9 was not counterbalanced by TIMP-1, it natural inhibitor, leading to the evident presence of the active MMP-9 enzyme in the traumatized nerve. Consistent with the enhancement of the proteases in the post-injury nerve, we recorded a significant increase in the excretion of the MMP activity in the urine of the CCI rats as compared with the control animals.

The sustained presence of the abnormal MMP-9 activity in the course of allodynia provides a biochemical means for the continued fragmentation of MBP and the release of its algesic epitope(s). The latter may lead to the generation of the specific autoantibodies circulating in the injured animals. To quantify this antibody pool we designed an ELISA methodology that employed the immobilized MBP84-104 peptide as bait. This ELISA allowed us to quantify the serum levels of the anti-peptide IgM and IgG antibodies. The serum level of the anti-algesic MBP84-104 IgM autoantibodies, but not IgG, continuously increased post-CCI, however, only in female rats. The upregulation of the IgM-type antibodies, the first antibodies type B cells produce in their response to an antigen, is likely relates to the short time-frame of the MBP84-104 epitope exposure in rats. It is well established that both the avidity and affinity of the pentameric IgM antibodies are superior relative to IgG. Importantly, the levels of the specific autoantibodies in female rats significantly exceeded that in male rats suggesting that there is sexual dimorphism in painful nociception and that the anti-MBP84-104 autoantibodies are not directly essential to allodynia. The unilateral nature of CCI-induced allodynia both in male and female rats supports this notion. These findings contribute to a better understanding of sex differences in pain and pain inhibition, and focus our attention on both autoimmunity and B cells which may contributes to pain syndromes primarily in females rather than in males (Shubayev et al. Neural Regen Res 11:890-891, (2016); Sorge et al. Nat Neurosci 18:1081-1083, (2015)).

Because the algesic MBP84-104 sequence is conserved in humans and rodents, the ELISA methodology we developed was also applicable to human serum samples. Our limited clinical study demonstrated that the levels of the autoantibodies against the algesic MBP fragment quantitatively discriminated the female subjects suffering from pain caused by a focalized myelin damage such as MS from female patients with a non-demyelinating pathology such as FMS.

In aggregate, this study provides the first evidence that the urinary protease activity and the circulating anti-algesic MBP autoantibodies accompany neuropathic pain-like behaviors in female rats. The ELISA methodologies we designed could be readily and inexpensively reproduced, and then used as a foundation for developing novel diagnostics for neuropathic pain in clinic, especially in women who are prevalent sufferers of chronic pain (Fillingim et al. J Pain 10:447-485, (2009); Nahin, J Pain 16:769-780, (2015)) and autoimmune conditions (Whitacre et al. Science 283:1277-1278, (1999)). Overall, our findings supports the hypothesis that adaptive immune system is a key in the development of mechanical allodynia (Sorge et al. Nat Neurosci 18:1081-1083, (2015)) and that mechanical allodynia represents an autoimmune condition related to myelin autoantigens, at least in females (Liu et al. J Neuroinflammation 9:119, (2012); Hong et al. Brain Behav Immun 60:282-292, (2017); Shubayev et al. Neural Regen Res 11:890-891, (2016)).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a detection agent” includes a plurality of such detection agents, reference to “the detection agent” is a reference to one or more detection agents and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different reporter agents does not indicate that the listed reporter agents are obvious one to the other, nor is it an admission of equivalence or obviousness.

Those skilled in the art will recognize, or be able to ascertain using no more than routine experimentation, many equivalents to the specific embodiments of the method and compositions described herein. Such equivalents are intended to be encompassed by the following claims.

We claim:

1. A method comprising treating a subject with a composition consisting essentially of an effective amount of a ligand for voltage-gated Ca²⁺-channel α2δ1 (CACNA2D1 ligand), wherein antibodies to myelin basic protein-derived peptide (MBP84-104) have been detected in the subject.

2. The method of claim 1 further comprising detecting the antibodies to MBP84-104 in the subject prior to treating the subject with the composition.

3. The method of claim 1 or 2, wherein the composition does not comprise a COX inhibitor, a sodium channel blocker, an NMDA antagonist, an opioid, or a non-steroidal anti-inflammatory drug (NSAID).

4. The method of any one of claims 1-3, wherein the subject is not treated with a COX inhibitor, a sodium channel blocker, an NMDA antagonist, an opioid, or a non-steroidal anti-inflammatory drug (NSAID).

5. The method of any one of claims 1-4, wherein the composition further comprises one or more pain relievers.

6. The method of any one of claims 1-5, wherein the subject is further treated with one or more pain relievers.

7. The method of any one of claims 1-6, wherein detection of the antibody to MBP84-104 in the subject indicates that the subject has a disease or condition that causes, or is associated with, the presence of, demyelination.

8. The method of claim 7, wherein the disease or condition is a demyelinating myelinoclastic disease or a demyelinating leukodystrophic disease.

9. The method of claim 7 or 8, wherein the disease or condition is inflammatory demyelination, viral demyelination, acquired metabolic demyelination, hypoxic-ischemic demyelination, or compression-induced demyelination.

10. The method of any one of claims 7-9, wherein the disease or condition is diabetic neuropathy, shingles, post herpetic neuralgia, neuromas, phantom limb pain, trigeminal neuralgia, multiple sclerosis, acute multiple sclerosis, neuromyelitis optica, concentric sclerosis, acute-disseminated encephalonyelitis, acute hemorrhagic leucoencephalitis, progressive multifocal leucoencephalopathy, human immunodeficiency virus infection, subacute sclerosing panencephalitis, central pontine myelinlysis, extrapontine myelinolysis, fibromyalgia, or complex regional pain syndrome.

11. The method of any one of claims 1-10, wherein the subject is suffering allodynia.

12. The method of any one of claims 1-11, wherein the subject is female.

13. The method of any one of claims 1-12, wherein the CACNA2D1 ligand is gabapentin or pregabalin.

14. A method comprising treating a subject with a pain reliever other than a composition consisting essentially of an effective amount of a ligand for voltage-gated Ca²⁺-channel α2δ1 (CACNA2D1 ligand), wherein antibodies to myelin basic protein-derived peptide (MBP84-104) were not detected in the subject.

15. The method of claim 14 further comprising determining the absence of antibodies to MBP84-104 in the subject prior to treating the subject with a pain reliever.

16. A method comprising refraining from treating a subject with a composition consisting essentially of an effective amount of a ligand for voltage-gated Ca²⁺-channel α2δ1 (CACNA2D1 ligand) if antibodies to myelin basic protein-derived peptide (MBP84-104) are not detected in the subject.

17. The method of claim 16 further comprising determining the absence of the antibodies to MBP84-104 in the subject prior to refraining from treating the subject with the composition.

18. The method of claim 16 further comprising treating the subject with one or more pain relievers if the antibodies to MBP84-104 are not detected in the subject.

19. A kit for detecting antibodies to myelin basic protein-derived peptide (MBP84-104) comprising:
a solid support, wherein MBP84-104 is immobilized on the solid support; and
a detection agent, wherein the detection agent comprises a detection element,
wherein detection of the detection element indicates the presence of the detection agent, wherein the presence of the detection agent indicates the presence of an antibody to MBP84-104.

20. The kit of claim 19, wherein the detection agent is an anti-antibody antibody, wherein the anti-antibody antibody is an anti-IgM antibody or an anti-IgG antibody.

21. The kit of claim 19 or 20, wherein the kit further comprises a reporter agent, wherein the reporter agent can facilitate detection of the detection element.

22. The kit of claim 21, wherein the anti-antibody antibody, the reporter agent, and the detection element are components of an enzyme-linked immunosorbent assay (ELISA) system.

23. The kit of any one of claims 19-22, wherein the detection element is an enzyme, wherein the enzyme catalyzes a reaction that can produce a detectable signal.

24. The kit of claim 23, wherein the reporter agent is an enzymatic substrate for the enzyme, wherein the enzyme can act on the reporter agent to produce the detectable signal.

25. The kit of any one of claims 19-24, wherein the solid support is in the form of a test strip.

26. The kit of claim 25, wherein the test strip is an immunochromatographic test strip.

27. A kit for detecting antibodies to myelin basic protein-derived peptide (MBP84-104) comprising:
one or more solid supports, wherein MBP84-104 is immobilized on at least one of the solid supports;
one or more antibodies, wherein at least one of the one or more antibodies is an antibody-detecting antibody, wherein each antibody-detecting antibody is independently an anti-IgM antibody or an anti-IgG antibody, wherein the antibody-detecting antibody comprises a detection element; and
a reporter agent, wherein the reporter agent can facilitate detection of the detection element, wherein detection of the detection element indicates the presence of the antibody-detecting antibody, wherein the presence of the antibody-detecting antibody indicates the presence of an antibody to MBP84-104.

28. A method of detecting the existence of demyelination in a subject, the method comprising:
bringing into contact a sample from the subject and the solid support of the kit of any one of claims 19-27 on which MBP84-104 is immobilized;
bringing into contact the solid support and the anti-antibody antibody;
bringing into contact the solid support and the reporter agent; and
detecting the presence of the reporter agent on the solid support,
wherein the reporter agent produces a detectable signal, wherein detection of the detectable signal on the solid support indicates the presence of the reported agent on the solid support, wherein detection of the reporter agent indicates the presence of the detection element on the solid support, wherein detection of the detection element indicates the presence of the anti-antibody antibody on the solid support, wherein detection of the anti-antibody antibody indicates the presence of an antibody to MBP84-104 in the sample.

29. A method of selecting a subject for treatment with a composition consisting essentially of an effective amount of a ligand for voltage-gated Ca²⁺-channel α2δ1 (CACNA2D1 ligand), the method comprising:
bringing into contact a sample from the subject and the solid support of the kit of any one of claims 19-27 on which MBP84-104 is immobilized;
bringing into contact the solid support and the anti-antibody antibody;
bringing into contact the solid support and the reporter agent;
detecting the presence of the reporter agent on the solid support,
wherein the reporter agent produces a detectable signal, wherein detection of the detectable signal on the solid support indicates the presence of the reported agent on the solid support, wherein detection of the reporter agent indicates the presence of the detection element on the solid support, wherein detection of the detection element indicates the presence of the anti-antibody antibody on the solid support, wherein detection of the anti-antibody antibody indicates the presence of an antibody to MBP84-104 in the sample; and
selecting the subject for treatment with the composition if the antibody to MBP84-104 is detected in the sample.

30. The method of claim 29 further comprising selecting the subject to not be treated with the composition if the antibody to MBP84-104 is not detected in the sample.

31. The method of any one of claims 28-30, wherein detection of the antibody to MBP84-104 in the sample indicates that the subject has a disease or condition that causes, or is associated with, the presence of, demyelination or neuropathic pain.

32. The method of claim 31, wherein the disease or condition is a demyelinating myelinoclastic disease or a demyelinating leukodystrophic disease.

33. The method of claim 31 or 32, wherein the disease or condition is inflammatory demyelination, viral demyelination, acquired metabolic demyelination, hypoxic-ischemic demyelination, or compression-induced demyelination.

34. The method of any one of claims 31-33, wherein the disease or condition is diabetic neuropathy, shingles, post herpetic neuralgia, neuromas, phantom limb pain, trigeminal neuralgia, multiple sclerosis, acute multiple sclerosis, neuromyelitis optica, concentric sclerosis, acute-disseminated encephalonyelitis, acute hemorrhagic leucoencephalitis, progressive multifocal leucoencephalopathy, human immunodeficiency virus infection, subacute sclerosing panencephalitis, central pontine myelinlysis, extrapontine myelinolysis, fibromyalgia, or complex regional pain syndrome.

35. The method of any one of claims 28-34, wherein the subject is suffering allodynia.

36. The method of any one of claims 28-35, wherein the subject is female.

37. The method of any one of claims 28-36, wherein the sample is a serum sample.

38. The method of any one of claims 28-37 further comprising administering an effective amount of the composition to the subject if the antibody to MBP84-104 is detected in the sample.

39. The method of claim 38, wherein the CACNA2D1 ligand is gabapentin or pregabalin.

40. The method of any one of claims 28-39 further comprising administering a pain reliever other than the composition to the subject if an antibody to MBP84-104 is not detected in the sample.

41. The method of any one of claims 28-39 further comprising refraining from administering the composition to the subject if the antibody to MBP84-104 is not detected in the sample.


SEQUENCE LISTING (12 sequences)

SEQ ID NO: 1 - protein, 31 aa, Homo sapiens
RTQDENPVVHFFKNIVTPRTPPPSQGKGRGL

SEQ ID NO: 2 - protein, 31 aa, Pan troglodytes; identical to SEQ ID NO: 1

SEQ ID NO: 3 - protein, 31 aa, Sus scrofa
RPQDENPVVHFFKNIVTPRTPPPSQGKGRGL

SEQ ID NO: 4 - protein, 31 aa, Cavia porcellus
RSQDENPVVHFFKNIVTPRTPPPSQGKGRGL

SEQ ID NO: 5 - protein, 31 aa, Rattus norvegicus; identical to SEQ ID NO: 1

SEQ ID NO: 6 - protein, 31 aa, Mus musculus
RTQDENPVVHFFKNIVTPRTPPPSQGKGGRD

SEQ ID NO: 7 - protein, 31 aa, Bos taurus; identical to SEQ ID NO: 3

SEQ ID NO: 8 - protein, 31 aa, Oryctolagus cuniculus
RPQDENPVVHFFKNIVTPRTPPPSQGKGRGT

SEQ ID NO: 9 - protein, 31 aa, Equus caballus; identical to SEQ ID NO: 3

SEQ ID NO: 10 - protein, 31 aa, Gallus
RPGDDNPVVHFFKNIVSPRTPPPMQAKGRGL

SEQ ID NO: 11 - protein, 21 aa, artificial sequence
  synthetic wild-type (MBP84-104-WT)
ENPVVHFFKNIVTPRTPPPSQ

SEQ ID NO: 12 - protein, 21 aa, Artificial Sequence
  scrambled (MBP84-104-SCR)
EFPHIKVTVVTPRNGFPNSPP